(19) United States
(12) Patent Application Publication
(10) Pub. No.: US 2024/0264173 A1
(43) Pub. Date: Aug. 8, 2024

(54) METHOD OF PROGNOSIS

(71) Applicant: PlaqueTec Ltd., Cambridge (GB)

(72) Inventor: Diane PROUDFOOT, Cambridge (GB)

(21) Appl. No.: 18/562,399

(22) PCT Filed: May 20, 2022
(86) PCT No.: PCT/GB2022/051281

§ 371 Date: Nov. 20, 2023

(30) Foreign Application Priority Data
May 21, 2021 (GB) 2107312.7

(51) Int. Cl. G01N 33/68

(52) U.S. Cl. CPC G01N 33/6893; G01N 2333/4753; G01N 2333/52; G01N 2800/26; G01N 2800/32; G01N 2800/52; G01N 2800/56

(57) ABSTRACT

Disclosed herein are methods and uses for identifying if a patient is predisposed to experiencing a severe episode of a disease and/or at risk of experiencing a severe episode of a disease, comprising detecting the presence of a biosignature comprising biomarkers as described herein. Also disclosed are methods of selecting medical interventions, evaluating the effectiveness of medical interventions, and of preventing and/or treating a severe episode of a disease, comprising detecting the presence of a biosignature comprising biomarkers as described herein and identifying if a patient is predisposed to experiencing a severe episode of a disease and/or at risk of experiencing a severe episode of a disease. Also disclosed are kits for identifying if a patient is predisposed to experiencing a severe episode of a disease and/or at risk of experiencing a severe episode of a disease, comprising a means for detecting the presence of a biosignature comprising biomarkers as described herein.

### FIELD OF INVENTION

The present invention relates to methods for identifying if a patient is predisposed to experiencing a severe episode of a disease and/or at risk of experiencing a severe episode of a disease, due to the presence of a biosignature comprising particular biomarkers. The present invention also relates to corresponding uses, methods of the treatment, and kits of parts.

### BACKGROUND

Challenges in managing the care of patients experiencing severe episodes of disease can lead to an increase in morbidity and mortality. Often patient care can be greatly improved if such episodes are predicted or expected, as it allows for earlier medical intervention. However, there is currently a lack of such predictive methods.

Using coronary artery disease as an example, it remains a major cause of death and disability worldwide. Although studies have identified individual circulating biomarkers associated with risk of major adverse coronary events (MACE) (Libby and Ebert, 2018. Circulation; 138:666-668; Tazaki et al., 2016, Int Heart J.; 57:18-24; and Chai et al., 2017, J Geriatr Cardiol.; 14:285-291), accurate prediction of their risk of future events is not currently possible (Piccolo et al., 2015, Lancet; 386:702-13 and Rossini et al., 2015. Catheter Cardiovasc Interv.; 85: E129-39).

As a further example, the Coronavirus disease 2019 (Covid-19) pandemic has provided the greatest challenge in modern medicine. As the pandemic has unfolded it has become apparent that different individuals suffer the disease at varying levels of severity, and often the health of individuals with Covid-19 can rapidly decline sometimes leading to death. The need to be able to identify which individuals are likely to be of most risk of suffering a severe episode of a disease is particularly pressing in a pandemic, where medical resources are stretched. However, it is currently very difficult to predict which individuals are likely to suffer most from Covid-19, making it very challenging to successfully manage patient care.

Therefore, to improve the management of patient care there is the need for methods for identifying if a patient is predisposed to, or at risk of, experiencing a severe episode of a disease. In particular, there is a need for minimally, or non-, invasive methods, as they are more appealing to patients than invasive methods, and often can be more simply undertaken without the need for complicated and expensive medical facilities.

Against this background, the inventors have discovered a number of biomarkers that are consistently, and clearly, detectable in blood samples from patients that are predisposed to, or at risk of, experiencing a severe episode of a disease. The inventors' work has identified that the biomarkers are detectable, and indicative, in patients with such a risk for a broad spectrum of diseases, including coronary artery disease and infectious diseases such as Covid-19 caused by severe acute respiratory syndrome coronavirus 2 (SARS-COV-2).

### BRIEF SUMMARY OF INVENTION

In a first aspect, the present disclosure provides a method for identifying if a patient is predisposed to experiencing a severe episode of a disease and/or at risk of experiencing a severe episode of a disease, comprising the steps of:
i) detecting the presence of a biosignature comprising the biomarkers hepatocyte growth factor (HGF) and Spondin-1 (SPON-1) in the patient's blood, and
ii) identifying that the patient is predisposed to experiencing a severe episode of a disease by the presence of the biosignature and/or identifying that the patient is at risk of experiencing a severe episode of a disease by the presence of the biosignature.

In a second aspect, the present disclosure provides a use of a biosignature comprising the biomarkers hepatocyte growth factor (HGF) and Spondin-1 (SPON-1) for identifying if a patient is predisposed to experiencing a severe episode of a disease and/or at risk of experiencing a severe episode of a disease, comprising:
i) detecting the presence of said biosignature comprising the biomarkers hepatocyte growth factor (HGF) and Spondin-1 (SPON-1) in the patient's blood, and
ii) identifying that the patient is predisposed to experiencing a severe episode of a disease by the presence of the biosignature and/or identifying that the patient is at risk of experiencing a severe episode of a disease by the presence of the biosignature.

In a third aspect, the disclosure provides a method of evaluating the effectiveness of a medical intervention in a patient, comprising the steps of:
a) detecting the presence of a biosignature comprising the biomarkers hepatocyte growth factor (HGF) and Spondin-1 (SPON-1) in the patient's blood at a first time,
b) applying the medical intervention to the patient,
c) detecting the presence of said biosignature comprising the biomarkers hepatocyte growth factor (HGF) and Spondin-1 (SPON-1) in the patient's blood at a second time, and
d) evaluating the effectiveness of the medical intervention by comparing the presence of the biosignature comprising the biomarkers in step a) with the presence of the biosignature comprising the biomarkers in step c).

In a fourth aspect, the disclosure provides a method of selecting a medical intervention for a patient, comprising the steps of:
i) detecting the presence of a biosignature comprising the biomarkers hepatocyte growth factor (HGF) and Spondin-1 (SPON-1) in the patient's blood,
ii) identifying that the patient is predisposed to experiencing a severe episode of a disease by the presence of the biosignature and/or identifying that the patient is at risk of experiencing a severe episode of a disease by the presence of the biosignature, and
iii) selecting a medical intervention for the patient.

In a fifth aspect, the disclosure provides a method of preventing and/or treating a severe episode of a disease in a patient, comprising the steps of:
i) detecting the presence of a biosignature comprising the biomarkers hepatocyte growth factor (HGF) and Spondin-1 (SPON-1) in the patient's blood,
ii) identifying that the patient is predisposed to experiencing a severe episode of a disease by the presence of the biosignature and/or identifying that the patient is at risk of experiencing a severe episode of a disease by the presence of the biosignature,
iii) selecting a medical intervention for the patient, and
iv) administering the medical intervention to the patient.

In a first particular embodiment of any of the aspects provided hereinabove, the method or use further comprises before step i) the step of:
i(i) providing a blood sample from the patient;
   wherein the detecting the presence of the biosignature in the patient's blood in step i) comprises detecting the presence of the biosignature in the sample of step i(i).

In second particular embodiment of any of the aspects provided hereinabove, the method further comprises before step (a) the step of:
a(i) providing a first blood sample from the patient; and
   wherein the detecting the presence of the biosignature in the patient's blood at a first time in step (a) comprises detecting the presence of the biosignature in the sample of step a(i); and wherein the method further comprises before step (c) the step of:
c(i) providing a second blood sample from the patient; and
   wherein the detecting the presence of the biosignature in the patient's blood at a second time in step (c) comprises detecting the presence of the biosignature in the sample of step c(i).

In a sixth aspect, the disclosure provides a kits of parts for identifying if a patient is predisposed to experiencing a severe episode of a disease and/or at risk of experiencing a severe episode of a disease, comprising a means for detecting the presence of a biosignature comprising three or more biomarkers selected from the list consisting of: hepatocyte growth factor (HGF); TNF-related weak inducer of apoptosis (TWEAK); Spondin-1 (SPON-1); tissue factor pathway inhibitor (TFPI); pappalysin-1 (PAPPA); CCL28; CXCL9; and azurocidin-1 (Azu-1); and C-reactive protein (CRP).

In a seventh aspect, the disclosure provides a method, use, or kit substantially as described herein.

### SUMMARY OF THE DRAWINGS

FIG. 1. Comparison of coronary and systemic protein levels—CVD1 protein panel (Olink)
A. Protein levels in plasma samples from 12 patients from the CS1 cohort, expressed as ratio of coronary upstream/peripheral(systemic) levels (CVD1 panel proteins on x-axis). Coronary samples upstream from an atherosclerotic plaque were collected using the PlaqueTec liquid biopsy system (LBS). Note 3 proteins indicated have high coronary/systemic protein ratios for 10 of the 12 patients.
B. As for A, showing coronary downstream/systemic protein ratios.
C. HGF absolute protein levels (NPX units) in coronary and peripheral (systemic) plasma, pre- (n=12) and post-PCI procedure (n=11).
D. Absolute levels of PAPPA (NPX units) in coronary and peripheral (systemic) plasma, pre- (n=12) and post-PCI procedure (n=11).
E. Absolute levels of SPON1 in coronary and peripheral (systemic) plasma, pre-(n=12) and post-PCI procedure (n=11). Note that 2 patients had much higher levels of HGF, PAPPA and SPON1 in peripheral samples prior to PCI compared with the other 10 patients, and levels of these proteins were higher in post-PCI samples.
F. Post-PCI/pre-PCI ratio of systemic samples (CVD1 panel). Growth Hormone (GH). Note similar patterns to A and B, indicating that for 10 of the 12 patients, conditions of the PCI procedure raised the systemic levels of these specific proteins. For 2 patients, these proteins were already at high levels systemically at baseline, before the procedure.

FIG. 2. Comparison of coronary and systemic protein levels repeat analysis of CS1 plasma in CVD3 and Inflammation protein panels (Olink)
A. The absolute (NPX) values for HGF in CVD1 panel. Abbreviations: PP—proximal port (coronary upstream), PS—peripheral (systemic) sample.
B. Repeatability measurements of HGF in Inflammation panel and comparison with healthy controls (age- and sex-matched for the CS1 cohort). Note that patterns for HGF measured on the CVD1 and Inflammation panels were similar, but NPX values differed between the 2 panels due to different dilution factors and dynamic ranges of the assays.
C. Coronary upstream:systemic protein ratios, Inflammation panel. Comparing with FIG. 1A, a similar HGF pattern was observed. Further proteins with similar patterns were identified: CXCL9, CCL28 and TWEAK.
D. Coronary upstream:systemic protein level ratios, CVD3 panel. Comparing with FIG. 1A, a similar but more distinct SPON1 pattern was observed. Further proteins with similar patterns were identified: TFPI and Azu1.

FIG. 3. CS1 patient and healthy control peripheral proteins levels

A. Individual patient absolute levels (NPX) for selected proteins using data from the CVD3 and Inflammation panels (both coronary and peripheral) were compared with 12 healthy, age- and sex-matched control plasma samples. These raw data plots revealed that levels of these proteins in coronary arteries of patients are clearly higher than healthy, age- and sex-matched control peripheral levels (n=12). 2 patients (patient 27 and 58) had a clear peripheral pattern of elevated HGF, SPON1, TFPI, CCL28, TWEAK and PAPPA. The same 2 patients also had elevated peripheral levels of CXCL9 and Azu-1 but 1 other patient in each case had even higher levels of these proteins. Additionally, healthy control values for CXCL9 and Azu-1 were very close to peripheral levels observed in patients 27 and 58. Therefore, although CXCL9 and Azu-1 were at raised levels in the 2 patients with the biosignature pattern, their profiles suggest they would not be useful in identification of individuals with the biosignature. (Note that PAPPA control levels were not determined as PAPPA is not included on the CVD3 and Inflammation panels).

B. No correlation of hsCRP with HGF levels Spearman R=−0.01 (P=0.99).

FIG. 4. Comparison of CS1 with PACIFIC cohort

Absolute levels (NPX) for selected peripheral proteins are displayed for CS1 cohort (left) and PACIFIC cohort (right). Patterns were identified, displaying similarities in the expression levels of the biosignature proteins, with 2 patients in each cohort displaying higher levels compared with the majority of patients. 2 participants (from 196 total) identified with the biosignature in the PACIFIC cohort are indicated (grey arrows). The extra dashed arrow in the CXCL9 plot displays one participant with higher levels of CXCL9 (but with low levels of the other biosignature proteins), confirming the observation made in CS1, that although CXCL9 appears to correlate with the biosignature, it may not be useful in identifying individuals with the biosignature.

FIG. 5. Comparison of protein values between cohorts and use for predicting absolute values of biosignature proteins.

A. For each of the cohorts analysed where raw data was available, the table displays median values for the whole cohort, minimum values for individuals identified with the biosignature and fold-changes between the median and biosignature minimum values for each protein.

B. Using published protein levels (mass/volume) by ELISA-based methods for HGF^{31,48,68-73}, SPON1^{31,74}, PAPPA^{75,76}, TWEAK^{31,77-80}, TFPI^{31,81-83}, CCL28^{31,84,85}, together with the fold-change definition of the biosignature, minimum thresholds were generated for potential use in identifying patients with the biosignature in future studies. Note that for CCL28, reported levels varied widely between studies, therefore caution should be taken when interpreting this average baseline level.

FIG. 6. Summary of biosignature prevalence and outcomes in different cohorts Using the biosignature definition: >90th centile and >1.5× median for HGF, PAPPA and SPON1 levels to identify participants with the biosignature, the table displays comparisons of biosignature prevalence and outcomes between cardiovascular disease cohorts, healthy controls, a respiratory disease/COVID-19 cohort and an ESKD/Covid-19 cohort. Non-PlaqueTec data (PACIFIC, respiratory disease/COVID-19 and ESKD COVID-19 cohorts) are from freely available data sources, while IMPROVE data was obtained in collaboration with Dr. Bruna Gigante. ¹Protein level data from SCAPIS (proteinatlas.org) was available in graph form, and ²Olink data was available as IQR values (insight.olink.com). Neither healthy dataset appeared to have individuals with the biosignature pattern. Individuals identified with the biosignature were in the minority, but a had higher incidence of CV events in IMPOVE and the respiratory disease/COVID-19 cohort, and a higher mortality in the respiratory disease/COVID-19 cohort and the ESKD/COVID-19 subcohort A compared with individuals without the biosignature. Abbreviations: CV, cardiovascular; CVD, cardiovascular disease; EBCT, electron beam computed tomography; ESKD, end-stage kidney disease; NA, not available.

FIG. 7. Survival outcomes for patients with the biosignature in the respiratory disease (COVID-19) cohort
A. Kaplan-Meier curve generated in GraphPad displaying probability of survival (%) in day 0 patients with the biosignature (11 patients) or without the biosignature (remaining 347 individuals).
B. The table in B shows the GraphPad statistical report. The log-rank test (Mantel-Cox) and the Gehan-Breslow-Wilcoxon test found statistical differences between the survival curves for each group (P<0.0001). Hazard ratio tests report that individuals die at 8.614 times (logrank) or 1065 times (Mantel-Haensel) the rate of individuals without the biosignature. As these hazard ratios are very different, hazard ratio calculations may not be useful for this dataset, likely due to the unbalanced groups and relatively small number of people in the biosignature group.

FIG. 8. Summary of functions of the biosignature proteins

### DETAILED DESCRIPTION OF INVENTION

One aspect of the present disclosure generally provides a method for identifying if a patient is predisposed to experiencing a severe episode of a disease and/or at risk of experiencing a severe episode of a disease, comprising the steps of: detecting the presence of a biosignature comprising the biomarkers hepatocyte growth factor (HGF) and Spondin-1 (SPON-1) in the patient's blood; and identifying that the patient is predisposed to experiencing a severe episode of a disease by the presence of the biosignature comprising the biomarkers and/or identifying that the patient is at risk of experiencing a severe episode of a disease by the presence of the biosignature comprising the biomarkers.

In a related aspect, the present disclosure generally provides use of a biosignature comprising the biomarkers hepatocyte growth factor (HGF) and Spondin-1 (SPON-1) for identifying if a patient is predisposed to experiencing a severe episode of a disease and/or at risk of experiencing a severe episode of a disease, comprising: detecting the presence of the biosignature in the patient's blood; and identifying that the patient is predisposed to experiencing a severe episode of a disease by the presence of the biosignature and/or identifying that the patient is at risk of experiencing a severe episode of a disease by the presence of the biosignature.

In embodiments of any of the methods and uses provided herein, the biosignature further comprises at least one or more biomarker selected from the group comprising or consisting of: TNF-related weak inducer of apoptosis (TWEAK); tissue factor pathway inhibitor (TFPI); pappalysin-1 (PAPPA); CCL28.

By “presence of the biosignature”, it is meant the presence of the biomarkers as described herein that are comprised by the biosignature. The terms “presence of the biosignature” and “presence of the biomarkers” are therefore used interchangeably in portions of the present disclosure.

The detecting the presence of the biosignature, as described herein, comprises detecting the presence of the biomarkers, as described herein. The presence of the biosignature identifies that the patient is predisposed to experiencing a severe episode of a disease and/or identifies that the patient is at risk of experiencing a severe episode of a disease. Accordingly, the presence of the biomarkers comprised by the biosignature also identifies that the patient is predisposed to experiencing a severe episode of a disease and/or identifies that the patient is at risk of experiencing a severe episode of a disease.

In one embodiment of the methods and uses provided by aspects of the present disclosure, a blood sample is provided from the patient. In embodiments where a blood sample is provided from the patient, the detecting the presence of the biomarker or biomarker, as described herein, in a patient's blood comprises detecting the presence of the biomarker or biomarkers, as described herein, in said blood sample from the patient.

In aspects of the present disclosure, methods and uses are provided comprising identifying that a patient is predisposed to experiencing a severe episode of a disease and/or at risk of experiencing a severe episode of a disease. The terms “predisposed to” and “at risk of” are generally terms of art that the person skilled in the art of medicine would readily understand.

As an exemplary definition, as used herein, where a patient is identified as being “predisposed to” experiencing a severe episode of a disease by the presence of the biosignature, as described herein, it is meant that the presence of said biosignature indicates that it is probable and/or likely that said patient will experience a severe episode of a disease. Accordingly, in one embodiment the patient is predisposed to experiencing a severe episode of a disease if it is probable and/or likely that said patient will experience a severe episode of a disease.

The predisposition could be due to one or more factors (which may be known or suspected) which include, in one embodiment of the invention: a genetic predisposition; a predisposition due to an existing episode of an illness and/or a previous episode of an illness (that illness may or may not be the disease the patient is predisposed to experiencing a severe episode of); and/or a predisposition caused by an exposure to an environmental stimulus and/or a stimulus external to the patient. For example, patients with cardiovascular disease (including patients exhibiting the biosignature) may be predisposed to experiencing a severe episode of a disease, wherein the disease is an infectious disease as described herein (including, for example, COVID-19).

As an exemplary definition, as used herein, where a patient is identified as being “at risk of” experiencing a severe episode of a disease by the presence of the biosignature, as described herein, it is meant that the presence of said biosignature indicates the patient has a probability and/or likelihood of experiencing a severe episode of a disease. Accordingly, in one embodiment the patient is at risk of experiencing a severe episode of a disease if it is probable and/or likely that said patient will experience a severe episode of a disease.

The “risk” may be due to no known or suspected predisposition and/or factor, other than the presence of the biosignature or the biomarkers, as described here.

In aspects and embodiments of the disclosure, the risk of experiencing a severe episode of a disease is an “elevated risk” of experiencing a severe episode of a disease. By “elevated level”, we include that the level (or measurement) of the biomarker(s) is higher than the level (or measurement) of the same biomarker(s) in the reference group of healthy individuals or general population.

In one embodiment, the probability or likelihood that the patient will experience a severe episode of the disease when the biosignature, as described herein, is present in said patient's blood or blood sample, is a higher and/or elevated probability and/or a higher and/or elevated likelihood than when compared to a reference group of individuals. In a preferred embodiment, the higher and/or elevated probability and/or a higher and/or elevated likelihood is statistically significant. It would be known to one skilled in the art of bioinformatics or statistics how to calculate such a statistically significance.

In a further embodiment, the reference group of individuals are healthy individuals that are known, or suspected, not to have the biomarkers, as described herein, in their blood or blood samples. It will be appreciated that such a reference group of healthy individuals that are known, or suspected, not to have the biomarkers in their blood or blood samples will not be identified as predisposed to experiencing a severe episode of a disease and/or identified as at risk of experiencing a severe episode of a disease. In some embodiments, the presence of the biosignature will not be identified in the reference group of healthy individuals that are known, or suspected, not to have the biomarkers, as described herein, in their blood or blood samples.

In one embodiment, the reference group of individuals are healthy individuals that are known, or suspected, to have the biomarkers, as described herein, in their blood or blood samples. It will be appreciated that such a reference group of healthy individuals that are known, or suspected, to have the biomarkers in their blood or blood samples will not be identified as predisposed to experiencing a severe episode of a disease and/or identified as at risk of experiencing a severe episode of a disease. In some embodiments, the presence of the biosignature will not be identified in the reference group of healthy individuals that are known, or suspected, to have the at biomarkers, as described herein, in their blood or blood samples.

In one embodiment the reference group of individuals are individuals who have a disease or illness, that are known, or suspected, to have the biomarkers, as described herein, in their blood or blood samples. The disease or illness may be the disease, as described herein. It will be appreciated that such a reference group of individuals who have a disease or illness, that are known, or suspected, to have the biomarkers in their blood will not be identified as predisposed to experiencing a severe episode of a disease and/or identified as at risk of experiencing a severe episode of a disease. In some embodiments, the presence of the biosignature will not be identified in the reference group of individuals who have a disease or illness that are known, or suspected, to have the biomarkers, as described herein, in their blood or blood samples.

In an alternative embodiment, the reference group of comparable individuals are individuals that are known to have the biomarkers, as described herein, in their blood or blood samples.

In a further embodiment, the reference group of individuals is the general population and/or a representative subgroup of the general population, wherein the subgroup has preferably been matched to the age, gender, ethnicity and/or nationality of the patient.

In a further embodiment, any reference group of individuals described herein has been matched to the age, gender, ethnicity and/or nationality of the patient, most preferably to the age and/or gender of the patient.

In one embodiment, the reference group of individuals or reference group of comparable individuals is one individual or one or more individuals.

In one embodiment, the probability and/or likelihood that the patient will experience a severe episode of a disease when the biosignature or biomarkers, as described herein, are present in said patient's blood or blood sample is the same probability and/or same likelihood as exists in the reference group of comparable individuals.

It will be appreciated that the presence of the biosignature—and hence the identification of a patient as being predisposed to experiencing a severe episode of a disease and/or at risk of experiencing a severe episode of a disease—may be detected by comparison of the levels of the biomarkers comprised by the biosignature in the patient's blood or the patient's blood sample to the level of the biomarkers in the blood samples of any reference group described herein.

Accordingly, in one embodiment, the biosignature is detected and a patient is identified as being predisposed to experiencing a severe episode of a disease by the presence in the patient's blood or the patient's blood sample of the biomarkers, as described herein, at about the, or is the, same level that said biomarkers, as described herein, are present in the blood samples of the individuals of a reference group of comparable individuals.

In one embodiment, the biosignature is detected and a patient is identified as predisposed to experiencing a severe episode of a disease by the presence in the patient's blood or the patient's blood sample of the biomarkers, selected from those as described herein, that are the same as are present in the blood or blood samples of the reference group of individuals or reference group of comparable individuals.

In one embodiment, the biosignature is detected and a patient is identified as being at risk of experiencing a severe episode of a disease by the presence in the patient's blood or the patient's blood sample of the biomarkers, selected from those as described herein, that are not present (or present at a low level) in the blood or blood samples of the reference group of healthy individuals or the general population.

In one preferred embodiment, the biosignature is detected and a patient is identified as being predisposed to experiencing a severe episode of a disease and/or at risk of experiencing a severe episode of a disease by the presence in the patient's blood or the patient's blood sample of elevated levels of the biomarkers, as described herein, compared to the level of said biomarkers, as described herein, that are present in the blood or blood samples of any reference group as described herein. In one embodiment, the biosignature is detected and a patient is identified as being predisposed to experiencing a severe episode of a disease and/or at risk of experiencing a severe episode of a disease by the presence in the patient's blood or the patient's blood sample of elevated levels of the biomarkers, as described herein, compared to the level of said biomarkers, as described herein, that are present in the blood or blood samples of the reference group of healthy individuals or general population. In one embodiment, the biosignature is detected and a patient is identified as being predisposed to experiencing a severe episode of a disease and/or at risk of experiencing a severe episode of a disease by the presence in the patient's blood or the patient's blood sample of elevated levels of the biomarkers, as described herein, compared to the level of biomarkers, as described herein, that are present in the blood or blood samples of the reference group of individuals who have a disease or illness.

In a particular embodiment of the invention, identifying if a patient is predisposed to experiencing a severe episode of a disease and/or at risk of experiencing a severe episode of a disease is providing a prognosis of the disease. The term “prognosis” would be known to one skilled in the art of medicine.

The methods and uses provided in aspects of the present disclosure comprise one or more steps comprising detecting the presence of hepatocyte growth factor (HGF) and Spondin-1 (SPON-1) in a patient's blood or a blood sample from a patient. In some embodiments, the methods and uses further comprise detecting the presence of one or more, optionally three or more, four or more, or five or more biomarkers selected from the list comprising or consisting of: TNF-related weak inducer of apoptosis (TWEAK); tissue factor pathway inhibitor (TFPI); pappalysin-1 (PAPPA); and CCL28 in a patient's blood or a blood sample from a patient.

As will be appreciated, the presence of any combination of the biomarkers described herein can be detected. For example, and by no limitation, in an embodiment in which three or more biomarkers are detected the three biomarkers could include: hepatocyte growth factor (HGF), TNF-related weak inducer of apoptosis (TWEAK), and Spondin-1 (SPON-1); or hepatocyte growth factor (HGF), TNF-related weak inducer of apoptosis (TWEAK), and tissue factor pathway inhibitor (TFPI); hepatocyte growth factor (HGF), Spondin-1 (SPON-1), and tissue factor pathway inhibitor (TFPI); or hepatocyte growth factor (HGF), Spondin-1 (SPON-1), and pappalysin-1 (PAPPA).

In a further preferred embodiment, the method or use comprises the step of detecting the presence of biomarkers comprising hepatocyte growth factor (HGF), and two or more biomarkers selected from the group comprising or consisting of: TNF-related weak inducer of apoptosis (TWEAK); Spondin-1 (SPON-1); tissue factor pathway inhibitor (TFPI); pappalysin-1 (PAPPA); and CCL28, in a patient's blood or a blood sample from a patient. In an alternative preferred embodiment, the method or use comprises the step of detecting the presence of biomarkers comprising hepatocyte growth factor (HGF) and TNF-related weak inducer of apoptosis (TWEAK), and one or more biomarkers selected from the group comprising or consisting of: Spondin-1 (SPON-1); tissue factor pathway inhibitor (TFPI); pappalysin-1 (PAPPA); and CCL28, in a patient's blood or a blood sample from a patient. In a further preferred embodiment, the method or use comprises the step of detecting the presence of at least hepatocyte growth factor (HGF), TNF-related weak inducer of apoptosis (TWEAK), and Spondin-1 (SPON-1) in a patient's blood or a blood sample from a patient.

It will be appreciated that the presence of any one of the biomarkers described herein may be detected in the absence of detecting of any one or all of the remaining biomarkers described herein. To put another way, detecting, say, three of the biomarkers as described herein but not the remaining four biomarkers in a patient will still lead to identifying that the patient is predisposed to experiencing a severe episode of a disease as, for example, outlined in the first aspect of the invention.

As used herein, the term “absence of detecting” may indicate that detecting of a biomarker or biomarkers, as described herein, was attempted, but the presence of said biomarker or biomarkers were not detected, or not detected at a sufficiently level high level, for example, when compared to a reference group of healthy individuals. Alternatively, the term “absence of detecting” may indicate that detecting of a biomarker or biomarkers, as described herein, was attempted, but detecting of the biomarker or biomarkers failed. The term “absence of detecting” may indicate that detecting of a biomarker or biomarkers, as described herein, was not attempted, and hence that said biomarker or biomarkers were not detected.

Detecting of a biomarker or biomarkers, as described herein, may fail due to error in performing the methods or uses provided herein. Types of error are known to the person skilled in the art. Detecting of a biomarker or biomarkers, as described herein, may fail due to the absence of said biomarker or biomarkers in a patient's blood or a blood sample from a patient. Accordingly, in some embodiments, the absence of detection of the biomarkers as described herein leads to the absence of detection of the biosignature as described herein.

In one embodiment, the method or use comprises the step of detecting the presence of hepatocyte growth factor (HGF) in the absence of detecting of the presence of one or more biomarkers selected from the list comprising or consisting of: TNF-related weak inducer of apoptosis (TWEAK); Spondin-1 (SPON-1); tissue factor pathway inhibitor (TFPI); pappalysin-1 (PAPPA); and CCL28, in a patient's blood or a blood sample from a patient.

In one embodiment, the method or use comprises the step of detecting the presence of TNF-related weak inducer of apoptosis (TWEAK) in the absence of detecting of the presence of one or more biomarkers selected from the list comprising or consisting of: hepatocyte growth factor (HGF); Spondin-1 (SPON-1); tissue factor pathway inhibitor (TFPI); pappalysin-1 (PAPPA); and CCL28, in a patient's blood or a blood sample from a patient.

In one embodiment, the method or use comprises the step of detecting the presence of Spondin-1 (SPON-1) in the absence of detecting of the presence of one or more biomarkers selected from the list comprising or consisting of: hepatocyte growth factor (HGF); TNF-related weak inducer of apoptosis (TWEAK); tissue factor pathway inhibitor (TFPI); pappalysin-1 (PAPPA); and CCL28, in a patient's blood or a blood sample from a patient.

In one embodiment, the method or use comprises the step of detecting the presence of tissue factor pathway inhibitor (TFPI) in the absence of detecting of the presence of one or more biomarkers selected from the list comprising or consisting of: hepatocyte growth factor (HGF); TNF-related weak inducer of apoptosis (TWEAK); Spondin-1 (SPON-1); pappalysin-1 (PAPPA); and CCL28, in a patient's blood or a blood sample from a patient.

In one embodiment, the method or use comprises the step of detecting the presence of pappalysin-1 (PAPPA) in the absence of detecting of the presence of one or more biomarkers selected from the list comprising or consisting of: hepatocyte growth factor (HGF); TNF-related weak inducer of apoptosis (TWEAK); Spondin-1 (SPON-1); tissue factor pathway inhibitor (TFPI); and CCL28, in a patient's blood or a blood sample from a patient.

In one embodiment, the method or use comprises the step of detecting the presence of CCL28 in the absence of detecting of the presence of one or more biomarkers selected from the list comprising or consisting of: hepatocyte growth factor (HGF); TNF-related weak inducer of apoptosis (TWEAK); Spondin-1 (SPON-1); tissue factor pathway inhibitor (TFPI); and pappalysin-1 (PAPPA), a patient's blood or a blood sample from a patient.

In one embodiment, the method or use comprises the step of detecting the presence of hepatocyte growth factor (HGF) and TNF-related weak inducer of apoptosis (TWEAK) in the absence of detecting of the presence of one or more biomarkers selected from the list comprising or consisting of: Spondin-1 (SPON-1); tissue factor pathway inhibitor (TFPI); pappalysin-1 (PAPPA); and CCL28, in a patient's blood or a blood sample from a patient. In one preferred embodiment, the method or use comprises the step of detecting the presence of hepatocyte growth factor (HGF), TNF-related weak inducer of apoptosis (TWEAK), and Spondin-1 (SPON-1) in the absence of detecting of the presence of one or more biomarkers selected from the list comprising or consisting of: tissue factor pathway inhibitor (TFPI); pappalysin-1 (PAPPA); and CCL28, in a patient's blood or a blood sample from a patient.

Accordingly, in one embodiment, the method or use comprises detecting the presence of hepatocyte growth factor (HGF) in a patient's blood or a blood sample from a patient. In one embodiment, the biosignature is detected and the patient is identified as being predisposed to experiencing a severe episode of a disease by the presence of hepatocyte growth factor (HGF) and Spondin-1 (SPON-1) and/or is identified as being risk of experiencing a severe episode of a disease by the presence of hepatocyte growth factor (HGF) and Spondin-1 (SPON-1).

In one embodiment, the method or use comprises detecting the presence of TNF-related weak inducer of apoptosis (TWEAK) in a patient's blood or a blood sample from a patient. In one embodiment, the biosignature is detected and the patient is identified as being predisposed to experiencing a severe episode of a disease by the presence of TNF-related weak inducer of apoptosis (TWEAK), hepatocyte growth factor (HGF) and Spondin-1 (SPON-1) and/or is identified as being risk of experiencing a severe episode of a disease by the presence of TNF-related weak inducer of apoptosis (TWEAK), hepatocyte growth factor (HGF) and Spondin-1 (SPON-1).

In one embodiment, the method or use comprises detecting the presence of tissue factor pathway inhibitor (TFPI) in a patient's blood or a blood sample from a patient. In one embodiment, the biosignature is detected and the patient is identified as being predisposed to experiencing a severe episode of a disease by the presence of tissue factor pathway inhibitor (TFPI), hepatocyte growth factor (HGF) and Spondin-1 (SPON-1) and/or is identified as being risk of experiencing a severe episode of a disease by the presence of tissue factor pathway inhibitor (TFPI), hepatocyte growth factor (HGF) and Spondin-1 (SPON-1).

In one embodiment, the method or use comprises detecting the presence of pappalysin-1 (PAPPA) in a patient's blood or a blood sample from a patient. In one embodiment, the biosignature is detected and the patient is identified as being predisposed to experiencing a severe episode of a disease by the presence of pappalysin-1 (PAPPA), hepatocyte growth factor (HGF) and Spondin-1 (SPON-1) and/or is identified as being risk of experiencing a severe episode of a disease by the presence of pappalysin-1 (PAPPA), hepatocyte growth factor (HGF) and Spondin-1 (SPON-1).

In one embodiment, the method or use comprises detecting the presence of CCL28 in a patient's blood or a blood sample from a patient. In one embodiment, the biosignature is detected and the patient is identified as being predisposed to experiencing a severe episode of a disease by the presence of CCL28, hepatocyte growth factor (HGF) and Spondin-1 (SPON-1) and/or is identified as being risk of experiencing a severe episode of a disease by the presence of CCL28, hepatocyte growth factor (HGF) and Spondin-1 (SPON-1).

In one embodiment, the biosignature further comprises the biomarker CXCL9. Accordingly, in one embodiment, the method or use comprises detecting the presence of CXCL9 in a patient's blood or a blood sample from a patient. This embodiment further comprises identifying that the patient is predisposed to experiencing a severe episode of a disease by the presence of CXCL9, hepatocyte growth factor (HGF) and Spondin-1 (SPON-1) and/or identifying that the patient is at risk of experiencing a severe episode of a disease by the presence of CXCL9, hepatocyte growth factor (HGF) and Spondin-1 (SPON-1).

In one embodiment, the biosignature further comprises the biomarker azurocidin-1 (Azu-1). Accordingly, in one embodiment, the method or use further comprises detecting the presence of the biomarker azurocidin-1 (Azu-1) in a patient's blood or a blood sample from a patient. This embodiment further comprises identifying that the patient is predisposed to experiencing a severe episode of a disease by the presence of Azu-1, hepatocyte growth factor (HGF) and Spondin-1 (SPON-1) and/or identifying that the patient is at risk of experiencing a severe episode of a disease by the presence of Azu-1, hepatocyte growth factor (HGF) and Spondin-1 (SPON-1).

The biosignature may comprise all of the biomarkers as described herein. Accordingly, in one embodiment the method or use further comprises detecting the presence of all of the hepatocyte growth factor (HGF); TNF-related weak inducer of apoptosis (TWEAK); Spondin-1 (SPON-1); tissue factor pathway inhibitor (TFPI); pappalysin-1 (PAPPA); and CCL28 in a patient's blood or a blood sample from a patient. In one embodiment, the method or use further comprises identifying that the patient is predisposed to experiencing a severe episode of a disease by the presence of the hepatocyte growth factor (HGF); TNF-related weak inducer of apoptosis (TWEAK); Spondin-1 (SPON-1); tissue factor pathway inhibitor (TFPI); pappalysin-1 (PAPPA); and CCL28 and/or identifying that the patient is at risk of experiencing a severe episode of a disease by the presence of the hepatocyte growth factor (HGF); TNF-related weak inducer of apoptosis (TWEAK); Spondin-1 (SPON-1); tissue factor pathway inhibitor (TFPI); pappalysin-1 (PAPPA); and CCL28.

In a preferred embodiment of the methods or uses provided by aspects of the disclosure, “detecting the presence” of a biomarker or biomarkers, as described herein, comprises detecting elevated, higher, or increased levels, values, or concentrations of a biomarker or biomarkers, as described herein, in a patient's blood or blood samples, compared to the level of said biomarker or biomarkers, as described herein, that is present in the blood or blood samples of any reference group as described herein.

Hepatocyte growth factor (HGF; UniProt ID: P14210) is a growth factor which is secreted by fibroblasts and has both angiogenic and mitogenic properties. Hepatocyte growth factor (HGF) plays a role in liver and kidney repair and in the development of bone metastases.

TNF-related weak inducer of apoptosis (TWEAK; UniProt ID: 043508) is a cytokine which functions in a number of signalling pathways. It is a member of the TNF superfamily and bind to the receptor Fn14. It signals through the NF-κB pathway and can stimulate a wide array of cytokines, chemokines and cell adhesion molecules. TWEAK plays a role in tissue inflammation, repair and regeneration in many diseases.

Spondin-1 (SPON-1; UniProt ID: Q9HCB6) is an extracellular matrix protein.

Tissue factor pathway inhibitor (TFPI; UniProt ID: P10646) is a protein that is involved in inhibiting tissue factor-induced coagulation. It is found in plasma lipoproteins and bound to the vascular endothelium. It binds to and inhibits factor Xa. The complex Xa-TFPI then interacts with the complex of tissue factor VIIa and inhibits its activation of factors X and IX.

Pappalysin-1 (PAPPA; UniProt ID: Q13219) is a metalloproteinase enzyme which is involved in bone formation, inflammation, wound healing and female fertility. The enzyme catalyses the cleavage of insulin-like growth factor binding proteins (IGFBPs). Once the IGFBP is cleaved, insulin growth factors dissociate from IGFBP and can then activate the IGF pathway by binding to the IGF receptors.

CCL28 (UniProt ID: Q9NRJ3) is a mucosa specific chemokine. It is expressed in epithelial cells from a variety of mucosal tissues, including salivary gland, trachea, colon, small intestine, rectum, and mammary gland. CCL28 is also known as mucosae-associated epithelial chemokine (MEC).

CXCL9 (UniProt ID: Q07325) is a cytokine which plays a role in the chemotaxis of immune cells. It is secreted by a number of immune and non-immune cells.

Azurocidin-1 (AZU-1; UniProt ID: P20160) is a neutrophil granule-derived antibacterial and monocyte- and fibroblast-specific chemotactic glycoprotein that also binds heparin.

We include that the biomarker or biomarkers, as described herein, are the protein or proteins, which, if present, would be found to circulate in the blood of the patient. In one embodiment, the biomarker(s) are the full-length protein(s) or fragment(s) of the full-length protein(s). It will be appreciated that detecting the fragment(s) of the full-length protein(s) can have utility as part of the invention if they can be identified as being derived or derivable from said full-length protein(s). One skilled in the art of medicine or biochemistry would be able to identify such fragment(s) as being relevant, using known analytical and bioinformatic methods.

In one embodiment of the methods and uses provided by aspects of the present disclosure, a blood sample is provided from the patient. In a preferred embodiment, the blood sample is a systemic blood sample, such as a peripheral blood sample. In one embodiment, the peripheral blood sample is a routine blood sample. In one embodiment, the peripheral blood sample is a baseline blood sample. In one embodiment, the peripheral blood sample does not comprise an anticoagulant (such as heparin—a so-called non-heparinised blood sample). The term peripheral blood would be known to one skilled in the art of medicine.

As an exemplary definition, as used herein, the term “peripheral blood sample” includes a sample of blood that is obtained from or obtainable from the peripheral vascular system (i.e., not the veins or arteries in the chest or abdomen of the patient).

In any of the aspects of the present disclosure, the blood sample may be obtained by, or obtainable by, one or more technique selected from the list consisting of: venepuncture; arteriopuncture; capillary sampling; fingerprick; heel-prick; scalp vein sampling; and ear lobe puncture. In one embodiment, the blood sample is obtained by or is obtainable by venepuncture.

In one embodiment of the methods and uses provided by aspects of the present disclosure, a blood sample is not provided from the patient.

In embodiments where a blood sample is not provided from the patient, the presence of a biomarker or biomarkers, as described herein, may be detected in the patient's blood within the patient's body. In one embodiment the presence of a biomarker or biomarkers, as described herein, is detected in blood vessels. In one embodiment, the blood vessels are peripheral blood vessels.

In one embodiment, the presence of a biomarker or biomarkers, as described herein, may be detected trans-dermally. Methods of trans-dermally detecting the presence of a biomarker and/or biomarkers, as described herein, are known to the person skilled in the art of diagnostics and/or medicine. Exemplary transdermal detection methods include infrared transdermal spectroscopy, optical transdermal imaging/detection, iontophoresis, microneedle-optofluidic biosensor detection, ultrasound, magnetic resonance imaging, and x-ray imaging.

In several aspects of the present disclosure, a patient is identified as being predisposed to experiencing a severe episode of a disease by the presence of a biosignature comprising biomarkers, as described herein, and/or identified as being at risk of experiencing a severe episode of a disease by the presence of a biosignature comprising biomarkers, as described herein.

In one embodiment, the disease is a disease associated with inflammation. In one embodiment, the disease associated with inflammation is characterised by symptoms resulting from inflammation and/or is a disease caused by inflammation. The inflammation may be caused by an illness (that illness may or may not be the disease the patient is predisposed to experiencing a severe episode of). In one embodiment, the illness is associated with an infection, such as an illness associated with a bacterial infection. In a preferred embodiment, inflammation caused by an illness associated with a bacterial infection is a result of a release of proteins from glycocalyx (which is a network of polysaccharides that can be found on the surface of some bacteria). In an alternative embodiment, the inflammation causes the disease. In one embodiment, the inflammation is vascular inflammation. In one embodiment, the inflammation is associated with endothelial damage (such as damage of endothelial cells). In one embodiment, the inflammation is associated with the activity of neutrophils and/or the activity of mast cells. Mast cells are an endogenous source of heparin and their activation and release of heparin may be one of the mechanisms responsible for elevated circulating levels of the biosignature proteins (da Silva E Z, et al. Mast cell function: a new vision of an old cell. J. Histochem. Cytochem. (2014) 62(10):698-738. doi: 10.1369/0022155414545334). Accordingly, in one embodiment, the methods and uses provided herein are not carried out where a heparin infusion has been administered to a patient. In some embodiments, the methods and uses provided herein are not carried out within 12 hours of administration of heparin to a patient, such as within 1 hour, 2 hours, 3 hours, 4 hours, 5 hours, 6 hours, 7 hours, 8 hours, 9 hours, 10 hours, 11 hours, or 12 hours of administration of heparin to a patient.

The disease may be a heritable disease. The disease may not be a heritable disease.

The person skilled in the art will understand how to determine whether a disease is associated with inflammation. The person skilled in the art will also understand methods that are appropriate for determining whether a patient or individual is experiencing inflammation.

In one embodiment, the disease is not associated with general systemic inflammation. In one embodiment, general systemic inflammation is characterised by the presence of the biomarker C-reactive protein (CRP). In one embodiment, the method or use as provided herein further comprises detecting the presence of the biomarker C-reactive protein (CRP) in a patient's blood or a blood sample from a patient. The method may further comprise identifying that the disease is not associated with general systemic inflammation by the presence of a low level of CRP. In one embodiment, a low level of CRP is a level (or value) of 5 mg/L or less, such as 4 mg/L or less, 3 mg/L or less, 2 mg/L or less, or 1 mg/L or less.

In embodiments where the method or use further comprises detecting the presence of the biomarker CRP, the step of detecting the presence of the biomarker CRP may be conducted prior to, concurrently with, or following any step of detecting the presence of the biosignature as provided herein. In some embodiments, the step of detecting the presence of the biomarker CRP is a routine CRP test. In some embodiments, the presence of the biomarker CRP may be an elevated level of CRP. By “elevated level of CRP” we include any level (or value) of CRP in a patient's blood or a blood sample from a patient that is above the average (including mean, median, or modal) level (or value) of CRP in the blood or blood samples from the individuals of a reference population or healthy population. The elevated level may be within clinically normal limits or may exceed a diagnostic threshold.

Accordingly, in some embodiments, the step of detecting the presence of the biosignature as disclosed herein—and subsequent steps of the methods and uses provided herein—is conducted if an elevated level of CRP has been detected in a patient's blood or blood sample from a patient. In some embodiments, the step of detecting the presence of the biosignature—and subsequent steps of the methods and uses provided herein—are not conducted if the level of CRP that is detected is not elevated.

General systemic inflammation as identified by detecting increased levels of CRP has been linked with increased risk of myocardial infarction and may be useful in stratifying patients most likely to benefit from targeted anti-inflammatory treatments.

Accordingly, in some embodiments, where a patient has been identified as being predisposed to experiencing a severe episode of a disease and/or identifying that a patient is at risk of experiencing a severe episode of a disease, an increased level of CRP may identify the patient as being more predisposed to experiencing a severe episode of the disease and/or may identify the patient as being at elevated risk of experiencing a severe episode of the disease, than if level of CRP was not increased.

C-reactive protein (CRP) (UniProt code: P02741) is a protein sometimes found in blood plasma, the concentration of which increases in response to general inflammation. CRP is induced, for example, by IL-1 and IL-6 signalling. C-reactive protein (CRP) binds moieties such as phosphocholine, phosphatidylcholine, and lysophosphatidylcholine on the surface of cells, thereby activating the complement system via C1q, the concentration of which increases greatly during acute phase response to tissue injury, infection or other inflammatory stimuli. The person skilled in the art of immunology and medicine would understand the functioning of the complement system.

In one embodiment, the disease is associated with endothelial dysfunction. The endothelial dysfunction may be as a consequence of the disease or may be a symptom of the disease. The endothelial dysfunction may be an aetiological factor of the disease. The endothelial dysfunction may be associated with any disease described herein. The person skilled in the art of medicine will understand the term “endothelial dysfunction”.

In one embodiment, the disease is a cardiovascular disease. Cardiovascular diseases are known to the person skilled in the art. Exemplary cardiovascular diseases include disease selected from the group comprising or consisting of: coronary heart disease, heart attack, congenital heart disease, hypertension, hypertensive heart disease, stroke (including ischemic stroke and hemorrhagic stroke), transient ischaemic attack (TIA), vascular dementia, angina (including stable angina and unstable angina), cerebrovascular disease, microvascular coronary disease, cardiomyopathy, heart failure, arrhythmias, vascular calcification, cardiac calcification, congestive heart failure, ishaemic heart disease, arrhythmia, coronary artery disease, deep vein thrombosis, pulmonary embolism, vascular embolism, cardiomyopathy, heart valve disease, pericardial disease, peripheral vascular disease, rheumatic heart disease, vascular disease, myocardial infarction, sudden cardiac death, inflammatory heart disease, carditis, endocarditis, myocarditis, eosinophilic myocarditis, aortic aneurysm, thromboembolic disease, stenosis, renal artery stenosis, erythromelalgia, Buerger's disease, Raynaud's disease, disseminated intravascular coagulation, and atherosclerosis.

In one embodiment, the disease is systemic lupus erythematosus or chronic kidney disease (such as end-stage kidney disease).

In one embodiment, the disease is an infectious disease. Exemplary infectious diseases include diseases associated with or caused by pathogens selected from the group comprising or consisting of: viruses, bacteria, fungi, and parasites. The pathogen may be an obligate pathogen, an opportunistic pathogen, or a zoonotic pathogen.

In one embodiment the disease is associated with a microbiome signature. As used herein, the term “microbiome” pertains to a microorganism or a plurality of microorganisms that colonise a location or organ on or within the human body. Exemplary locations and/or organs include but are not limited to: gastrointestinal tract, skin, eyes, conjunctiva, hair, urethra, bladder, vagina, placenta, uterus, mouth, lung, and biliary tract. The microbiome signature may be the presence or absence of a microorganism or a plurality of microorganisms. Exemplary microorganisms may be selected from the group comprising or consisting of: viruses, bacteria, fungi, and parasites. The microbiome signature may be dysbiosis. The microbial signature may be associated with inflammation. In one embodiment in which the disease is associated with a microbiome signature, the disease is Crohn's disease or ulcerative colitis.

The virus may be a DNA virus or an RNA virus, such as a double-stranded DNA (dsDNA) virus, a single-stranded DNA (ssDNA) virus, a double-stranded RNA (dsRNA) virus, or a single-stranded RNA (ssRNA) virus. The virus may be an enveloped or non-enveloped virus. In one embodiment, the virus is a virus or viral pathogen selected from the group comprising or consisting of: a coronavirus, a flavivirus, or a poxvirus. In one embodiment, the virus or viral pathogen is selected from the group comprising or consisting of: SARS-COV-1, SARS-COV-2, MERS-COV, dengue virus (DENV), west nile virus (WNV), yellow fever virus (YFV), Japanese encephalitis virus (JEV), Zika virus (ZIKV), Tick-borne encephalitis virus (TBEV), and vaccina virus (VV). Accordingly, the disease may be COVID-19. Infection with a coronavirus (for example, SARS-COV-2), or COVID-19, may lead to the patient developing Long COVID.

The bacteria may be a gram-positive bacterium or a gram-negative bacterium. In one embodiment, the bacteria is elected from the group comprising or consisting of: Actinobacteria, Bacteroidetes, Firmicutes, Chlamydiae, Proteobacteria, and Spirochaetes. In one embodiment, the bacterium is a bacterium belonging to a genus selected from the group comprising or consisting of: Escherichia, Klebsiella, Mycobacterium, Staphylococcus, and Streptococcus. Accordingly, the disease may be selected form diseases including but not limited to: gastroenteritis; urinary tract infection (UTI); neonatal meningitis; meningitis; haemolytic-uremic syndrome; peritonitis; mastitis; septicaemia; pneumonia; sepsis; ankylosing spondylitis; spondyloarthropathy; tuberculosis; stye; abscesses; boils; carbuncles; folliculitis; ear infection; impetigo; cellulitis; osteomyelitis; septic arthritis; asthma; rhinitis; sinusitis; endocarditis; necrotising pneumonia; necrosis; necrotising fasciitis; toxic shock syndrome; scaled skin syndrome; paronychia; eczema; strep throat; pink eye; and erysipelas.

The fungus may be a filamentous fungus, a hyphal fungus, or a yeast. In one embodiment, the fungus belongs to the genus Candida. Accordingly, the disease may be selected from diseases including but not limited to: candidiasis; thrush; fungemia; and onychomycosis.

The parasite may be an alveolate parasite, an amoeban parasite, an apicomplexan parasite, a parasitic flatworm, a kinetoplastid parasite, a nematode parasite, a protozoan parasite, a parasitic roundworm, or a tapeworm. In one embodiment, the parasite is a parasite that belongs to a genus selected from the group comprising or consisting of: Plasmodium. Accordingly, the disease may be Malaria.

In one embodiment, the patient is experiencing an episode of the disease. In one embodiment, the patient is exhibiting symptoms of the disease, in particular one or more symptoms of the disease.

To put another way, in this embodiment in which the patient is experiencing an episode of the disease, the disease may be confirmed or already diagnosed. Methods of confirming a disease or diagnosing a patient with a disease are known the person skilled in the art medicine (for example, the patient is exhibiting one or more symptoms of the disease and/or diagnostic test show that the patient has the disease, and from that information the person skilled in the art of medicine would be able to confirm that the patient has the disease). In an alternative embodiment, the disease may be suspected, as could be identified as such by the person skilled in the art of medicine (for example, the patient is exhibiting one or more symptoms of the disease and/or diagnostic test show that the patient has the disease, but from that information the person skilled in the art of medicine is not able to confirm the patient has the disease). In this embodiment, the methods and uses described herein are of particular use in identifying if a patient is predisposed to experiencing a severe episode of a disease and/or at risk of experiencing a severe episode of the confirmed disease.

In one embodiment, the patient is not exhibiting symptoms of the disease. To put another way, the disease is not suspected. Where a disease is suspected or is not suspected, a patient may be asymptomatic or pre-symptomatic of said disease.

In the embodiment of the invention in which the disease is suspected and/or is not suspected, the methods and/or uses of the present invention can be, or are, used to diagnose the disease and/or are used as part of the diagnosis of the disease.

Symptoms and signs of the diseases as described herein would be known to the person skilled in the art of medicine. Exemplary symptoms of cardiovascular disease and infectious diseases including bacterial infection, fungal infection, and infection with a parasite include those set out in Wilkinson et al. (2017) Oxford Handbook of Clinical Medicine (10^{th} Ed), Oxford University Press.

The disease may be associated with old age. The disease may be a geriatric disease. The patient may be a geriatric patient. Accordingly, the patient may be aged over about 40, over about 50, over about 60, over about 70, over about 80, over about 90, or over about 100. The patient may have a disease that is associated with old age or that is a geriatric disease, but the patient may not be a geriatric patient. Accordingly, the patient may have an early-onset variant or form of a disease associated with old age or a geriatric disease. The patient may therefore be under about 100, under about 90, under about 80, under about 70, under about 60, under about 50, under about 40, under about 30, under about 20, or under about 10 years of age. The age of the patient with the disease associated with old age or a geriatric disease may be younger than the average age of onset of said disease in a group or population of individuals or patients with said disease.

The disease may be associated with the activity of immune cells. Immune cells are known to the person skilled in the art; however, exemplary immune cells may be selected from the group comprising or consisting of: basophils, B-cells or B lymphocytes, dendritic cells, eosinophils, γδ T cells, granulocytes, innate lymphoid cells, macrophages, mast cells, monocytes, natural killer (NK) cells, neutrophils, and T-cells or T lymphocytes. Accordingly, in one embodiment, the disease is associated with the activity of neutrophils and/or mast cells. The activity of immune cells, including neutrophils and/or mast cells, may be elevated in a patient or individual who has the disease compared to a patient or individual who does not have the disease. The activity of immune cells, including neutrophils and/or mast cells, may be decreased in a patient or individual who has the disease compared to a patient or individual who does not have the disease. It will be appreciated that the activity of immune cells, including neutrophils and/or mast cells, may be elevated or decreased as a consequence of the disease as defined herein (for example, as a consequence of infection with a virus, bacterium, fungus, or parasite as defined herein); or may comprise part of the aetiology of the disease.

Methods of determining or measuring immune cell activity are known in the art. For instance, an exemplary method of identifying mast cell activation may comprise detecting an increased mast cell burden in whole blood counts and detecting an elevated serum or plasma level of tryptase (e.g., above about 11 ng/ml tryptase in serum or plasma) (Akim, 2017, J. Allergy Clin. Immunol., 140(2):349-355 doi: 10.1016/j.jaci.2017.06.007. PMID: 28780942). An exemplary method of identifying neutrophil activation may comprise detecting increased levels of myeloperoxidase (MPO) and/or neutrophil elastase in plasma (e.g., when compared to control levels of MPO and/or neutrophil elastase in control plasma) and/or detecting enlarged neutrophil cells in plasma (e.g., when compared to the size of control neutrophils in control plasma) (Perdomo et al., 2019, Nat. Commun., 10:1322 doi: 10.1038/s41467-019-09160-7).

Accordingly, in some embodiments, the methods or uses as provided herein further comprise detecting tryptase, and/or myeloperoxidase, neutrophil elastase or proteins indicative of glycocalyx damage (e.g., syndecan-1) in a patient's serum, plasma, or blood; or in a serum sample, a plasma sample, or blood sample from a patient. The tryptase, myeloperoxidase, and/or neutrophil elastase may be detected concurrently with, prior to, or after any of the steps of the methods or uses provided herein. In some embodiments, the biosignature further comprises as biomarkers tryptase, myeloperoxidase (MPO), and/or neutrophil elastase.

As used herein, a severe episode of a disease is characterised by the patient needing a medical intervention to recover from the episode of a disease and/or the patient being at risk of death due to the episode of the disease.

In one embodiment, the medical intervention comprises hospitalisation. The medical intervention may comprise surgery. Exemplary surgeries may be selected from the group comprising or consisting of: amputation, angiography, atherectomy, balloon angioplasty, balloon emblectomy, catheter ablation, cardiac cauterisation, cardioverter defibrillator implant, carotid endarterectomy, carotid-subclavian bypass, catheterisation, Cimino fistula, coronary artery bypass surgery, coronary artery stent implant, DRIL, endarterectomy, endovascular aneurysm repair, dialysis catheter, endovenous laser treatment, heart transplant, hybrid arch debranching, inferior vena cava filter implant, intra-aortic balloon pump implant, intubation, kidney transplant, laparoscopic kidney surgery, laser angioplasty, MILLER banding, nephrectomy (including partial nephrectomy and radical nephrectomy), open aortic surgery, pacemaker implant, minimally invasive heart surgery, percutaneous coronary intervention, revision using distal inflow, sclerotherapy, stenting, surgical decompression, surgical revascularization, sympathectomy, thoracic endovascular aneurysm repair, thrombectomy, tracheotomy, valvuloplasty, valve repair valve replacement, vascular assist device implant, vascular bypass surgery, vein stripping, or any combination thereof. The medical intervention may comprise a non-surgical therapeutic intervention. Exemplary non-surgical therapeutic interventions may be selected from the group comprising or consisting of: cardiopulmonary resuscitation, defibrillation, percussion, ventilation, postural drainage, proning, haemodialysis, peritoneal dialysis, hemofiltration, hemodiafiltration, or any combination thereof.

The medical intervention may comprise administration of at least one drug or pharmaceutical compound to the patient. Exemplary drugs or pharmaceutical compounds may be selected from the group comprising or consisting of: ACE inhibitors, angiotensin AT1 receptor blockers, angiotensin-converting enzyme inhibitors, angiotensin-(1-7), angiotensin II receptor agonists, antibiotics or antibacterial drugs or agents (including but not limited to: aminoglycosides, ansamycins, carbacephems, carbapenems, cephalosporins, glycopeptides, lincosamides, lipopeptides, macrolides, monobactams, nitrofurans, oxazolidinones, penicillins, polypeptides, quinolones, fluoroquinolones, sulfonamides, and tetracyclines, such as drugs selected from the group comprising or consisting of: amikacin, dibekacin, gentamicin, kanamycin, neomycin, netilmicin, plazomicin, sisomicin, tobramycin, paromomycin, streptomycin, spectinomycin, rifaximin, loracarbef, biapenem, doripenem, ertapenem, imipenem or cilastatin, lenapenem, meropenem, panipenem/betamipron, razupenem, tomopenem, thienamycin, tebipenem, cefacetrile, cefadroxil, cefalexin, cefaloglycin, cefalonium, cefaloridine, cefalotin, cefapirin, cefatrizine, cefazaflur, cefazedrone, cefazolin, cefradine, cefroxadine, ceftezole, cefaclor, cefonicid, cefprozil, cefuroxime, cefuzonam, cefmetazole, cefoxitin, cefotetan, cefbuperazone, cefminox, cefotiam, cefcapene, cefdaloxime, cefdinir, cefditoren, ceftamet, cefixime, cefmenoxime, cefodizime, cefovecin, cefpimizole, cefpodoxime, cefteram, ceftibuten, ceftiofur, ceftiolene, ceftizoxime, ceftriaxone, cefoperazone, ceftazidime, latamoxef, cefclidine, cefepime, cefidorocol, cfluprenam, cefoselis, cefozopram, cefpirome, cefquinome, flomoxef, ceftobiprole, ceftaroline, ceftaroline fosamil, ceftolozane, cefaloram, cefaparole, cefcanel, cefedrolor, cefempidone, cefetrizole, cefivitril, cefmatilen, cefmepidium, cefoxazole, cefrotil, cefsumide, ceftioxide, cefuracetime, cefaphrin, cefalothin, cefamandole, cefotaxime, moxalactam, cefsulodin, teicoplanin, vancomycin, telavancin, dalbavancin, oritavancin, clindamycin, lincomycin, pirlimycin, daptomycin, surfactin, azithromycin, clarithromycin, erythromycin, fidamoxicin, roxithromycin, telithromycin, spiramycin, fidaxomicin, carbomycin, josamycin, midecamycin, oleandomycin, solithromycin, troleandomycin, tylosin, roxithromycin, cethromycin, solithromycin, aztreonam, nocardicin A, tigemonam, difurazone, furazolidone, nifufoline, nifuroxazide, nifurquinazol, nifurtoinol, nifurzide, nitrofural, nitrofurantoin, ranbezolid, contezolid, cycloserine, linezolid, posizolid, radezolid, tadezolid, torezolid, amoxicillin, ampicillin, azlocillin, bacampicillin, benzypenicillin, benethamine penicillin, benzathine penicillin, carbenicillin, carfecillin, ciclacillin, clavulanic acid, cloxacillin, docloxacillin, epicillin, flucloxacillin, hetacillin, mecillinam, mezlocillin, metampicillin, methicillin, nafcillin, oxacillin, penicillin F, penicillin X, penicillin K, penicillin G, penicillin V, phenethicillin, phenoxy-methyl penicillin, pirmecillinan, piperacillin, pivampicillin, procaine penicillin, sulbactam, talampicillin, tazobactam, temocillin, ticarcillin, bacitracin, colistin, polymyxin B, sodium fusidate, bleomycin, cinoxacin, ciprofloxacin, enoxacin, flumequine, fleroxacin, gatifloxacin, gemifloxacin, levofloxacin, lomefloxacin, mafenide, moxifloxacin, nadifloxacin, nalidixic acid, norfloxacin, ofloxacin, oxolinic acid, trovafloxacin, grepafloxacin, rosoxacin, sparfloxacin, temafloxacin, pefloxacin, rufloxacin, balofloxacin, pazufloxacin, clinafloxacin, moxifloxacin, sitafloxacin, prulifloxacin, besifloxacin, delafloxacin, ozenoxacin, danofloxacin, difloxacin, enrofloxacin, ibafloxacin, marbofloxacin, ordifloxacin, sarafloxacin, sulfacetamide, sulfadiazine, silver sulfadiazine, sulfadimidine, sulfamethoxazole, sulfanilimide, sulfasalazine, sulfisoxazole, sulfonamidochrysoidine, sulfasomidine, sulfamoxole, sulfanitran, sulfadimethoxine, sulfamethoxypyridazine, sulfametoxydiazine, sulfadoxine, terephtyl, tetracycline, clomocycline, chlortetracycline, oxytetracycline, demeclocycline, lymecycline, meclocycline, methacycline, minocycline, rolitetracycline, doxycycline, tigecycline, eravacycline, sarecycline, omadacycline, clofazimine, dapsone, cycloserine, capreomycin, ethambutol, ethionamide, isoniazid, pyrazinamide, rifampicin, rifabutin, rifapentine, arsphenamine, chloramphenicol, fosfomycin, fusidic acid, metronidazole, mupirocin, platensumycin, thiamphenicol, tinidazole, trimethoprim, teixobactin, malacidin, friulimicin, halicin, demeclocydine, framycetin, calcium sulphaloxate, sulfametopyrazine, sulphaguanidine, sulphaurea, hexamine, and colistimethate, and the like), antibodies, anticoagulants, antihistamines (including but not limited to: azelastine, brompheniramine, carbinoxamine, cetirizine, cyproheptadine, chlorpheniramine, desloratadine, diphenhydramine, fexofenadine, hydroxyzine, levocabastine, levocetirizine, loratadine, and the like), antihypertensive drugs, antifungal drugs or agents (including but not limited to: polyenes, azoles (including imidazoles, triazoles, and thiazoles), allylamines, echinocandins, aurenes, benzoic acid, ciclopirox, flucytosine, griseofulvin, haloprogin, tolnaftate, undecylenic acid, tracetin, crystal violet, castellani's paint, ortomide, miltefosine, potassium iodide, nikkomycin, coal tar, copper(II) sulfate, selenium disulfide, sodium thiosulfate, piroctone olamine, iodoquinol, clinoquinol, acrisorcin, zinc pyrithione, sulfur, and the like), antiviral drugs or agents (including but not limited to: abacavir, acyclovir, adefovir, amantadine, ampligen, amprenavir, umifenovir, atazanavir, atripla, baloxavir marboxil, baricitinab, bamlanivimab, biktarvy, boceprevir, bulvirtide, bulevirtide, casirivimab, cidofovir, cobicistat, combivir, daclatasvir, darunavir, delavirdine, descovy, didanosine, docosanol, dolutegravir, doravirine, edoxudine, efavirenz, elvitegravir, emtricitabine, enfuvirtide, entrcavir, etesevimab, etravirine, famciclovir, favipiravir, fomivirsen, fosamprenavir, foscarnet, ganciclovir, ibacitabine, ibalizumab, idoxuidine, imiquimod, imunovir, indinavir, imdevimab, lamivudine, letermovir, lipinavir, lopinavir, loviride, maraviroc, methisazone, moroxydine, nelfinavir, nevirapine, nxavir, nitazoxanide, norvir, oseltamivir, penciclovir, permivir, pleconaril, podophyllotoxin, raltegravir, REGN-COV2, remdesivir, ribavirin, rilpivirine, rimantadine, ritonavir, saquinavir, simeprevir, sofosbuvir, stavudine, taribavirin, telaprevir, telbivudine, tenofovir alafenamide, tenofir disoproxil, tipranavir, triazavirin, trifluridine, trizivir, tromantadine, Truvada, umfenovir, unifenovir, valaciclovir, valganciclovir, vicriviroc, vidarabine, zalcitabine, zanamivir, and zidovudine, and the like), antiparasitic drugs or agents (including, antiprotozoals, antihelminthics, antinematode drugs, anticestodes, antitrematodees, antiamoebics, such as drugs selected from the group comprising or consisting of: abamectin, albendazole, amodiaquine, artemisinin, aremether, arteether, atesunate, atovaquone, benzimidazole, chloroquine, clindamycin, derquantel, diethylcarbamazine, dihydroartemisinin, doxycycline, eflornithine, emodepside, fenbendazole, flubendazole, furazolidone, halofantrine, hydroxychloroquine, ivermectin, lumefantrine, levamisole, mebendazole, mefloquine, melarsoprol, metronidazole, monepantel, niclosamide, nifursemizone, nitazoxanide, octadepsipeptide, ornidazole, oxyclozanide, paromomycin, pentamidine, praziquantel, proguanil, primaquine, pyrantel pamoate, pyrimethamine, quinapyramine, quinine, ronidazole, salicylanilide, spiroindole, sulfonamide, suramin, tinidazole, thiabendazole, triclabendazole, and the like), anistreplase, alteplase, antioxidants, azathioprine, azithromycin, belimumab, beta blockers, bradykinin, budesonide, calcium channel blockers (including dihydropyridine calcium channel blockers such as amlopidine, azelnidipine, bendipine, israpidine, nicardipine, nifedipine), calcitriol, corticosteroids, coumarin, cyclophosphamide, dexamethasone, endothelial NO synthase enhancers (including AVE3085), factor Xa inhibitors, flavonoids, heparin, hydroxychloroquine, I_{f} inhibitors (e.g., ivrabadine), intravenous immunoglobulins, interferons, mast cell stabilisers (including but not limited to sodium cromoglicate, ketotifen, vitamin D, quercetin, chromoglycic acid, luteolin, and the like; and antibodies targeting Siglec-8 such as lirentelimab and the like), phosphodiesterase-5 inhibitors, phosphate binders, polyphenols, potassium, prednisone, methotrexate, mycophenolate, non-steroidal anti-inflammatory drugs, opioid drugs, reteplase, sphingosine-1-phosphate, statins, steroids, streptokinase, tacrolimus, tencteplase, thrombin inhibitors, tocilizumab, urokinase, vitamin C, dexamethasone, warfarin, zinc, or any combination thereof.

In one embodiment, the severe episode of a disease is a major adverse coronary event (MACE). Exemplary MACEs may be selected from the group comprising or consisting of: heart failure, ischemic cardiovascular events, myocardial infarction, stroke, revascularisation, or any combination thereof.

In one embodiment, the severe episode of a disease results in death.

Aspects and embodiments of the methods and uses provided herein comprise steps of detecting the presence of a biomarker or biomarkers as described herein. Methods of determining the level of a biomarker or biomarkers, as described herein, are well-known to those skilled in the art, such as proteomic methods.

As used herein, the term “proteomic methods” relate to methods that may be employed to measure protein quantitatively or qualitatively, preferably quantitively. The person skilled in the art would understand which proteomic methods would be appropriate to employ in the methods and uses provided herein.

Exemplary proteomic methods may be selected from the group comprising or consisting: any antibody-based detection method, Enzyme Linked Immunosorbent Assay (ELISA), Polyacrylamide Gel Eletrophoresis (PAGE), Sodium Dodecylsulfate (SDS)-PAGE, 2D-SDS-PAGE, western blot, northwestern blot, southwestern blot, Proximity Extension Assay (PEA), immunohistochemical staining two-dimensional chromatography (2D-LC), high-performance liquid chromatography (HPLC), partition chromatography, partition chromatography, hydrophilic interaction chromatography (HILIC), normal-phase chromatography (NP-HPLC), displacement chromatography, reversed-phase chromatography (RC-HPLC), size-exclusion chromatography (SEC), ion-exchange chromatography, anion-exchange chromatography (AEX-HPLC), bioaffinity chromatography, aqueous normal-phase chromatography, tandem liquid-chromatography-mass spectrometry (LC-MS), mass spectrometry (MS), matrix-assisted laser desorption/ionization (MALDI), MALDI-MS, atmospheric pressure MALDI (AP-MALDI), time-of-flight MALDI (MALTI-TOF), MALDI-FT-ICR, and any combination thereof.

Measurements produced by proteomic methods are terms of art and are well-known to the person skilled in the art. Proteomic measurements may be absolute or relative and may be the absolute mass or weight of protein; protein concentration; or relative level of protein. Absolute mass or weight of protein may be expressed in pg, ng, μg, mg, g, or kg. Absolute mass or weight of protein may be expressed in moles, for example pmol, nmol, μmol, mmol, or mol. Protein concentration may be expressed as the mass or weight of protein in a given volume of a solvent, wherein a given volume of solvent is expressed in pL, nL, μL, mL, dL, cL, or L. For example, protein concentration may be expressed as pg/mL. Protein concentration may be expressed as the molarity of said protein, for example pM, nM, μM, mM, or M. Relative protein level measurements may include fold change, fold difference, fold increase, fold decrease, normalised protein level, or normalized protein expression (NPX).

Relative protein levels may be relative to a reference absolute or relative measurement of protein, for example a reference mass, weight, concentration, or relative level of protein. The reference level of protein may be determined from any reference group of individuals as defined herein.

Measurements produced by proteomic methods may also include the indication of the presence or absence of a protein, such as the presence or absence of a protein in a sample. The presence or absence of a protein may be absolute (i.e., the presence of a protein may be detected or may not be detected); or may be relative to a threshold level of said protein.

The person skilled in the art will understand which proteomic methods are suitable for producing which proteomic measurements. For example, Proximity Extension Assay (PEA) analysis is suitable for producing measurements comprising normalised protein expression (NPX) measurements of protein level; and ELISA analysis is suitable for producing measurements comprising concentration (for example, pg/mL) measurements of protein level. The person skilled in the art will understand how to derive other measurements, for example fold-change, by calculation from absolute and/or relative measurements, for example from NPX measurements or concentration measurements of protein level.

In aspects and embodiments of the methods and uses provided herein, the presence of the biosignature is detected and a patient is identified as being predisposed to experiencing a severe episode of a disease by the presence of the biomarkers, as described herein, and/or identified as being at risk of experiencing a severe episode of a disease by the presence of the biomarkers as described herein.

In one embodiment, detecting the presence of the biomarker or biomarkers comprises detecting the level of said biomarker or biomarkers above a certain threshold (such as an absolute value or a relative value).

In one embodiment, the presence of the biosignature is detected and the patient is identified as being predisposed to experiencing a severe episode of a disease and/or at risk of experiencing a severe episode of a disease if the biomarkers, as described here, are at an absolute level (or value) that is higher than a baseline level of the biomarkers. In one embodiment, the baseline level (or value) is the level (or value) of the biomarkers, as described herein, in a reference group of healthy individuals or the general population. In one embodiment, the baseline level (or value) is the median level (or value) of a biomarker or biomarkers, as described herein, in a reference group of healthy individuals. In a further embodiment, the baseline level (or value) is the level (or value) of the biomarker or biomarkers, as described herein, in a reference group of individuals who have a or the disease or illness. In a further embodiment, the baseline level (or value) is the median level (or value) of a biomarker or biomarkers, as described herein, in a reference group of individuals who have a or the disease or illness. In a further embodiment, the baseline level (or value) is the known level (or value) of a biomarker or biomarkers as identifiable in scientific literature and/or as derivable from the data in the Examples. In a preferred embodiment, the higher level (or value) is statistically significant. The absolute value may be a mass or weight, or a concentration. Based on the information described here, such absolute levels would be apparent to one skilled in medicine.

Exemplary baseline levels (or values) of a biomarker or biomarkers, as described herein, in a reference group of healthy individuals or the general population are given herein. Accordingly, in one embodiment, the baseline level (or value) of hepatocyte growth factor (HGF) is about 246.0 NPX. In one embodiment, the baseline level (or value) of TNF-related weak inducer of apoptosis (TWEAK) is about 673.0 NPX. In one embodiment, the baseline level (or value) of Spondin-1 (SPON-1) is about 1.6 NPX. In one embodiment, the baseline level (or value) of tissue factor pathway inhibitor (TFPI) is about 682.0 NPX. In one embodiment, the baseline level (or value) of CCL28 is about 3.4 NPX.

Exemplary baseline levels (or values) of a biomarker or biomarkers, as described herein, in a reference group of individuals who have a or the disease or illness are given herein. Accordingly, in one embodiment, the baseline level (or value) of hepatocyte growth factor (HGF) is about 245.0 NPX, about 402.5 NPX, about 539.5 NPX, about 1212.9, about 977.2 NPX, about 1727.0 NPX, or about 3414.5 NPX. In one embodiment, the baseline level (or value) of TNF-related weak inducer of apoptosis (TWEAK) is about 118.3 NPX, about 406.8 NPX, about 446.3 NPX, about 417.3 NPX, about 520.0 NPX, about 673.0 NPX, or about 938.7 NPX. In one embodiment, the baseline level (or value) of Spondin-1 (SPON-1) is about 0.9 NPX, about 1.5 NPX, about 2.7 NPX, about 3.0 NPX, about 3.2 NPX, about 4.1, or about 40.8 NPX. In one embodiment, the baseline level (or value) of tissue factor pathway inhibitor (TFPI) is about 70.9 NPX, about 384.0 NPX, about 664.1 NPX, about 730.5 NPX, about 741.1, about 860.3, or about 793.2 NPX. In one embodiment, the baseline level (or value) of pappalysin-1 (PAPPA) is about 4.0 NPX, about 4.2 NPX, about 5.6 NPX, about 12.8 NPX, about 14.7 NPX, or about 16.2 NPX. In one embodiment, the baseline level (or value) of CCL28 is about 2.1 NPX, about 3.3 NPX, about 9.5 NPX, about 10.4 NPX, about 11.1 NPX, or about 13.5 NPX.

Further exemplary baseline levels are given herein. In one embodiment, the baseline level (or value) of hepatocyte growth factor (HGF) is about 339 pg/mL. In one embodiment, the baseline level (or value) of hepatocyte growth factor (HGF) is about 335 pg/mL. In one embodiment, the baseline level (or value) of TNF-related weak inducer of apoptosis (TWEAK) is about 521 pg/mL. In one embodiment, the baseline level (or value) of TNF-related weak inducer of apoptosis (TWEAK) is about 377 pg/mL. In one embodiment, the baseline level (or value) of tissue factor pathway inhibitor (TFPI) is about 69 pg/mL. In one embodiment, the baseline level (or value) of tissue factor pathway inhibitor (TFPI) is about 73.4 ng/ml. In one embodiment, the baseline level (or value) of pappalysin-1 (PAPPA) is about 23 ng/ml. In one embodiment, the baseline level (or value) of Spondin-1 (SPON-1) is about 15 ng/ml. In one embodiment, the baseline level (or value) of CCL28 is about 234 ng/ml. In one embodiment, the baseline level (or value) of CCL28 is about 782 pg/mL.

The level of a biomarker may be a relative level of a biomarker.

In one embodiment, the presence of the biosignature is detected and the patient is identified as being predisposed to experiencing a severe episode of a disease and/or at risk of experiencing a severe episode of a disease if the level (or value) of the biomarkers comprised by the biosignature are one and a half-fold or more higher, preferably when compared to a baseline level, such as: a two-fold or more, a three-fold or more, a four-fold or more, a five-fold or more, a six-fold or more, a seven-fold or more, an eight-fold or more, a nine-fold or more, a ten-fold or more, an 11-fold or more, a 12-fold or more, a 13-fold or more, a 14-fold or more, a 15-fold or more, a 16-fold or more, a 17-fold or more, and 18-fold or more, a 19-fold or more, or a 20-fold or more higher.

In one embodiment, the biosignature comprises hepatocyte growth factor (HGF) and the patient is identified as being predisposed to experiencing a severe episode of a disease and/or at risk of experiencing a severe episode of a disease if the level (or value) of hepatocyte growth factor (HGF) is a fold change of one and a half or more higher, preferably when compared to a baseline level, such as: a two-fold or more, a three-fold or more, a four-fold or more, a five-fold or more, a six-fold or more, a seven-fold or more, an eight-fold or more, a nine-fold or more, a ten-fold or more, an 11-fold or more, a 12-fold or more, a 13-fold or more, a 14-fold or more, a 15-fold or more, a 16-fold or more, a 17-fold or more, and 18-fold or more, a 19-fold or more, or a 20-fold or more change higher. In preferred embodiment, the level (or value) of hepatocyte growth factor (HGF) is a fold change of 2.2 or more higher, 2.4 or more higher, 3.4 or more higher, 3.9 or more higher, 4.7 or more higher, 5.0 or more higher, 18.2 or more higher, or 19.0 or more higher.

In one embodiment, the biosignature comprises TNF-related weak inducer of apoptosis (TWEAK) and the patient is identified as being predisposed to experiencing a severe episode of a disease and/or at risk of experiencing a severe episode of a disease if the level (or value) of TNF-related weak inducer of apoptosis (TWEAK) is a fold change of one and a half or more higher, preferably when compared to a baseline level, such as: a two-fold or more, a three-fold or more, a four-fold or more, a five-fold or more, a six-fold or more, a seven-fold or more, an eight-fold or more, a nine-fold or more, a ten-fold or more, an 11-fold or more, a 12-fold or more, a 13-fold or more, a 14-fold or more, a 15-fold or more, a 16-fold or more, a 17-fold or more, and 18-fold or more, a 19-fold or more, or a 20-fold or more change higher. In preferred embodiments, the level (or value) of TNF-related weak inducer of apoptosis (TWEAK) is a fold change of 1.5 or more higher, 2.3 or more higher, 2.7 or more higher, 3.3 or more higher, 3.4 or more higher, 3.7 or more higher, 5.7 or more higher, or 10.5 or more higher.

In one embodiment, the biosignature comprises Spondin-1 (SPON-1) and the patient is identified as being predisposed to experiencing a severe episode of a disease and/or at risk of experiencing a severe episode of a disease if the level (or value) of Spondin-1 (SPON-1) is a fold change of one and a half or more higher, preferably when compared to a baseline level, such as: a two-fold or more, a three-fold or more, a four-fold or more, a five-fold or more, a six-fold or more, a seven-fold or more, an eight-fold or more, a nine-fold or more, a ten-fold or more, an 11-fold or more, a 12-fold or more, a 13-fold or more, a 14-fold or more, a 15-fold or more, a 16-fold or more, a 17-fold or more, and 18-fold or more, a 19-fold or more, or a 20-fold or more change higher. In preferred embodiments, the level (or value) of Spondin-1 (SPON-1) is a fold change of 1.7 or more higher, 2.0 or more higher, 2.2 or more higher, 2.5 or more higher, 2.8 or more higher, 3.0 or more higher, 5.9 or more higher, 6.1 or more higher.

In one embodiment, the biosignature comprises tissue factor pathway inhibitor (TFPI) and the patient is identified as being predisposed to experiencing a severe episode of a disease and/or at risk of experiencing a severe episode of a disease if the level (or value) of tissue factor pathway inhibitor (TFPI) is a fold change of one and a half or more higher, preferably when compared to a baseline level, such as: two-fold or more, a three-fold or more, a four-fold or more, a five-fold or more, a six-fold or more, a seven-fold or more, an eight-fold or more, a nine-fold or more, a ten-fold or more, an 11-fold or more, a 12-fold or more, a 13-fold or more, a 14-fold or more, a 15-fold or more, a 16-fold or more, a 17-fold or more, and 18-fold or more, a 19-fold or more, or a 20-fold or more change higher. In preferred embodiments, the level (or value) of tissue factor pathway inhibitor (TFPI) is a fold change of 1.4 or more higher, 1.7 or more higher, 1.9 or more higher, 2.2 or more higher, 2.9 or more higher, 3.0 or more higher, or 3.2-fold or more higher.

In one embodiment, the biosignature comprises is pappalysin-1 (PAPPA) and the patient is identified as being predisposed to experiencing a severe episode of a disease and/or at risk of experiencing a severe episode of a disease if the level (or value) of pappalysin-1 (PAPPA) is a fold change of one and a half or more higher, preferably when compared to a baseline level, such as: a two-fold or more, a three-fold or more, a four-fold or more, a five-fold or more, a six-fold or more, a seven-fold or more, an eight-fold or more, a nine-fold or more, a ten-fold or more, an 11-fold or more, a 12-fold or more, a 13-fold or more, a 14-fold or more, a 15-fold or more, a 16-fold or more, a 17-fold or more, and 18-fold or more, a 19-fold or more, or a 20-fold or more change higher. In preferred embodiments, the level (or value) of pappalysin-1 (PAPPA) is a fold change of 2.1 or more higher, 2.4 or more higher, 3.1 or more higher, 3.9 or more higher, 4.8 or more higher, 5.2 or more higher, 5.3 or more higher, 7.8 or more higher.

In one embodiment, the biosignature comprises CCL28 and the patient is identified as being predisposed to experiencing a severe episode of a disease and/or at risk of experiencing a severe episode of a disease if the level (or value) of CCL28 is a fold change of is one and a half or more higher, preferably when compared to a baseline level, such as: a two-fold or more, a three-fold or more, a four-fold or more, a five-fold or more, a six-fold or more, a seven-fold or more, an eight-fold or more, a nine-fold or more, a ten-fold or more, an 11-fold or more, a 12-fold or more, a 13-fold or more, a 14-fold or more, a 15-fold or more, a 16-fold or more, a 17-fold or more, and 18-fold or more, a 19-fold or more, or a 20-fold or more change higher. In preferred embodiments, the level (or value) of CCL28 is a fold change of 1.4 or more higher, 1.9 or more higher, 2.1 or more higher, 2.7 or more higher, 3.4 or more higher, 3.5 or more higher, 4.2 or more higher.

The threshold may be determined by comparison of the level of the biomarker or biomarkers, as described herein, in blood or a blood sample or blood samples from a patient or a population of patients identified as being predisposed to experiencing a severe episode of a disease and/or at risk of experiencing a severe episode of a disease with the level of a biomarker or biomarkers, as described herein, in blood or a blood sample or blood samples from a reference group of individuals as defined herein. The threshold may be determined by comparison of the level of a biomarker or biomarkers, as described herein, in blood or a blood sample or blood samples from a patient or a population of patients who have experienced a severe episode of a disease with the level of a biomarker or biomarkers, as described herein, in blood or a blood sample or blood samples from reference group of individuals or as defined herein.

Accordingly, in one embodiment, the biosignature comprises hepatocyte growth factor (HGF) and the patient is identified as being predisposed to experiencing a severe episode of a disease and/or at risk of experiencing a severe episode of a disease if the hepatocyte growth factor (HGF) is above a certain threshold. In one embodiment, the hepatocyte growth factor (HGF) is above a threshold of about 2000 NPX, for example about 2709 NPX, about 3763 NPX, about 4666 NPX, about 5474.0 NPX, about 5920.4 NPX, about 7319 NPX, or about 8353.0 NPX. In one embodiment, the hepatocyte growth factor (HGF) is above a threshold of about 1199 NPX. In one embodiment, the hepatocyte growth factor (HGF) is above a threshold of 1695 pg/mL. In one embodiment, the hepatocyte growth factor (HGF) is above a threshold of 502.5 pg/mL.

In one embodiment, the biosignature comprises TNF-related weak inducer of apoptosis (TWEAK) and the patient is identified as being predisposed to experiencing a severe episode of a disease and/or at risk of experiencing a severe episode of a disease if the TNF-related weak inducer of apoptosis (TWEAK) is above a certain threshold. In one embodiment, the TNF-related weak inducer of apoptosis (TWEAK) is above a threshold of about 1000 NPX, for example about 1007.5 NPX, about 1388 NPX, about 5318 NPX, or about 5474 NPX. In one embodiment, the TNF-related weak inducer of apoptosis (TWEAK) is above a threshold of about 179.7 NPX, about 417.3 NPX, or about 673.0 NPX. In one embodiment, the TNF-related weak inducer of apoptosis (TWEAK) is above a threshold of 1042 pg/mL. In one embodiment, the TNF-related weak inducer of apoptosis (TWEAK) is above a threshold of 565.5 pg/mL.

In one embodiment, the biosignature comprises Spondin-1 (SPON-1) and the patient is identified as being predisposed to experiencing a severe episode of a disease and/or at risk of experiencing a severe episode of a disease if Spondin-1 (SPON-1) is above a certain threshold. In one embodiment, the Spondin-1 (SPON-1) is above a threshold of about 5 NPX, for example about 5.6 NPX, about 6.2 NPX, about 7.6 NPX, about 8.9 NPX, about 9.2 NPX, about 114.3 NPX, or about 120.6 NPX. In one embodiment, the Spondin-1 (SPON-1) is above a threshold of 53.5 ng/ml.

In one embodiment, the biosignature comprises tissue factor pathway inhibitor (TFPI) and the patient is identified as being predisposed to experiencing a severe episode of a disease and/or at risk of experiencing a severe episode of a disease if tissue factor pathway inhibitor (TFPI) is above a certain threshold. In one embodiment, the tissue factor pathway inhibitor (TFPI) is above a threshold of about 1000 NPX, for example about 1029 NPX, about 1118 NPX, about 1512 NPX, about 1613 NPX, about 1677 NPX, or about 2151 NPX. In one embodiment, the tissue factor pathway inhibitor (TFPI) is above a threshold of 138 pg/mL. In one embodiment, the tissue pathway inhibitor (TFPI) is above a threshold of 110.1 ng/ml.

In one embodiment, the biosignature comprises pappalysin-1 (PAPPA) and the patient is identified as being predisposed to experiencing a severe episode of a disease and/or at risk of experiencing a severe episode of a disease if pappalysin-1 (PAPPA) is above a certain threshold. In one embodiment, the pappalysin-1 (PAPPA) is above a threshold of about 8 NPX, for example about 11.7 NPX, about 13.5 NPX, about 19.1 NPX, about 32.9 NPX, about 40 NPX, about 63.2 NPX, about 77.2 NPX, or about 77.3 NPX. In one embodiment, the pappalysin-1 (PAPPA) is above a threshold of 46 ng/ml. In one embodiment, the pappalysin-1 (PAPPA) is above a threshold of 53.7 ng/ml.

In one embodiment, the biosignature comprises CCL28 and the patient is identified as being predisposed to experiencing a severe episode of a disease and/or at risk of experiencing a severe episode of a disease if CCL28 is above a certain threshold. In one embodiment, the CCL28 is above a threshold of about 8 NPX, for example about 8.8 NPX, about 11.3 NPX, about 15.9 NPX, about 20 NPX, about 20.1 NPX, about 30 NPX, about 35.2 NPX, or about 46.2 NPX. In one embodiment, the CCL28 is above a threshold of 1644 pg/mL. In one embodiment, the CCL28 is above a threshold of 1173 pg/mL. In one embodiment, the CCL28 Is above a threshold of 351 ng/ml.

In one embodiment, biomarker level (NPX) is determined by Proximity Extension Assay (PEA) analysis of a patient's blood sample. In one embodiment, biomarker concentration (pg/mL) is determined by ELISA analysis of a patient's blood sample.

In one embodiment, the presence of the biosignature is detected and the patient is identified as being predisposed to experiencing a severe episode of a disease and/or at risk of experiencing a severe episode of a disease if the levels (or value) of hepatocyte growth factor (HGF) and Spondin-1 (SPON-1) in the patient's blood or blood sample are above a threshold value as provided herein. In one embodiment, the presence of the biosignature is detected and the patient is identified as being predisposed to experiencing a severe episode of a disease and/or at risk of experiencing a severe episode of a disease if the levels (or value) of hepatocyte growth factor (HGF), Spondin-1 (SPON-1), and one or more biomarkers selected from the group comprising or consisting of: TNF-related weak inducer of apoptosis (TWEAK), tissue factor pathway inhibitor (TFPI), pappalysin-1 (PAPPA), and/or CCL28 in the patient's blood or blood sample are above a threshold value as provided herein.

In one embodiment, the presence of the biosignature is detected and the patient is identified as being predisposed to experiencing a severe episode of a disease and/or at risk of experiencing a severe episode of a disease if the levels (or value) of hepatocyte growth factor (HGF) and Spondin-1 (SPON-1) in the patient's blood or blood sample are elevated by a fold change above baseline value as provided herein. In one embodiment, the presence of the biosignature is detected and the patient is identified as being predisposed to experiencing a severe episode of a disease and/or at risk of experiencing a severe episode of a disease if the levels (or value) of hepatocyte growth factor (HGF), Spondin-1 (SPON-1), and one or more biomarkers selected from the group comprising or consisting of: TNF-related weak inducer of apoptosis (TWEAK), tissue factor pathway inhibitor (TFPI), pappalysin-1 (PAPPA), and/or CCL28 in the patient's blood or blood sample are elevated by a fold change above baseline value as provided herein.

In one embodiment, the presence of the biosignature is detected and the patient is identified as being predisposed to experiencing a severe episode of a disease and/or at risk of experiencing a severe episode of a disease if the level (or value) of hepatocyte growth factor (HGF) has a fold change of five or more higher than the median level (or value), and the levels (or values) of at least two other biomarkers have a fold change of two or more higher than the median levels (or values). In one embodiment, the presence of the biosignature is detected and the patient is identified as being predisposed to experiencing a severe episode of a disease and/or at risk of experiencing a severe episode of a disease if the level (or value) of hepatocyte growth factor (HGF) has a fold change of five or more higher than the median level (or value), and the levels (or values) of at least two other biomarkers have a fold change of two or more higher than the median levels (or values), and the patient has a low level (or value) c-reactive protein (CRP). Low C-reactive protein (CRP) may be defined as a CRP concentration 5 mg/L or less, as discussed herein. The median value may be the median value of the same biomarker or biomarkers in a reference group of healthy individual or the general populations.

In one embodiment, the presence of the biosignature is detected and the patient is identified as being predisposed to experiencing a severe episode of a disease and/or at risk of experiencing a severe episode of a disease if the level (or value) of the biomarkers comprised by the biosignature are above the 85^{th} percentile, for example above the 86^{th} percentile, above the 87^{th} percentile, above the 88^{th} percentile, above the 89^{th} percentile, above the 90^{th} percentile, or higher in the patient's blood or a sample of blood from the patient. The percentile may be calculated from and/or compared to the levels of the biomarker or biomarkers in a reference group of healthy individuals or the general population. In one embodiment, the presence of the biosignature is detected and the patient is identified as being predisposed to experiencing a severe episode of a disease and/or at risk of experiencing a severe episode of a disease if the level (or value) of the biomarkers comprised by the biosignature in the patient's blood or a sample of blood from the patient are above a percentile as described above, and above one and a half times the median level (or value) of said biomarkers in in a reference group of healthy individuals or the general population. In one embodiment, the presence of the biosignature is detected and the patient is identified as being predisposed to experiencing a severe episode of a disease and/or at risk of experiencing a severe episode of a disease if the level (or value) of the biomarkers comprised by the biosignature in the patient's blood or a sample of blood from the patient are above the 85^{th} percentile or higher, and above one and a half times the median level (or value) of said biomarkers in in a reference group of healthy individuals or the general population. In a preferred embodiment, the presence of the biosignature is detected and the patient is identified as being predisposed to experiencing a severe episode of a disease and/or at risk of experiencing a severe episode of a disease if the level (or value) of the biomarkers comprised by the biosignature in the patient's blood or a sample of blood from the patient are above the 90^{th} percentile or higher, and above one and a half times the median level (or value) of said biomarkers in a reference group of healthy individuals or the general population. In one embodiment, the biomarkers are hepatocyte growth factor (HGF), Spondin-1 (SPON-1), and optionally any other biomarker as defined herein. In one preferred embodiment, the biomarkers are hepatocyte growth factor (HGF), Spondin-1 (SPON-1), Pappalysin-1 (PAPPA), and optionally any other biomarker as defined herein.

It will be appreciated that, using the methods, uses, and kits as described herein, a patient may be identified as not being predisposed to experiencing a severe episode of a disease by the presence of a biomarker or biomarkers, as defined herein, and/or identified as not being at risk of experiencing a severe episode of a disease by the presence of a biomarker or biomarkers, as defined herein. A patient may be identified as not being predisposed to experiencing a severe episode of a disease by the presence of a biomarker or biomarkers, as defined herein, and/or identified as not being at risk of experiencing a severe episode of a disease by the presence of a biomarker or biomarkers, as defined herein, if the presence of a biomarker or biomarkers are detected at a level (or value) below a given fold change or threshold level of said biomarker or biomarkers as described hereinabove.

In some embodiments, the presence of biosignature may not be detected and hence a patient may be identified as being healthy—i.e., the biosignature may be absent. A patient may be identified as being healthy if the level (or value) of a biomarker or biomarkers, as defined herein, are below a given fold change, preferably when compared to a baseline level. A patient may be identified as being healthy if the level (or value) of Hepatocyte growth factor (HGF) is a fold change of about 1.6 or below. A patient may be identified as being healthy if the level (or value) of TNF-related weak inducer of apoptosis (TWEAK) is a fold change of about 1.2 or below. A patient may be identified as being healthy if the level (or value) of Spondin-1 (SPON-1) is a fold change of about 1.2 or below. A patient may be identified as being healthy if the level (or value) of tissue factor pathway inhibitor (TFPI) is a fold change of about 1.2 or below. A patient may be identified as being healthy if the level (or value) of CCL28 is a fold change of about 1.4 or below.

A patient may be identified as being healthy if the level (or value) of a biomarker or biomarkers, as defined herein, are below a given threshold. A patient may be identified as being healthy if the level (or value) of Hepatocyte growth factor (HGF) is about 401.0 NPX or below. A patient may be identified as being healthy if the level (or value) of Spondin-1 (SPON-1) is about 1.9 NPX or below. A patient may be identified as being healthy if the level (or value) of CCL28 is about 4.6 NPX or below.

It will be appreciated that—where a patient is identified as being predisposed to experiencing a severe episode of a disease by the presence of the biosignature comprising the biomarkers, as defined herein, and/or identifying that the patient is at risk of experiencing a severe episode of a disease by the presence of the biosignature comprising the biomarkers, as defined herein—it may be desirable to apply a medical intervention that prevents the patient from experiencing a severe episode of a disease, decreases the risk that the patient experiences a severe episode of a disease, or mitigates, ameliorates, or palliates the effects, symptoms, or patient's experience of a severe episode of a disease.

In certain embodiments, the method or use comprises the step of selecting a medical intervention for the patient. Exemplary medical interventions may be selected from any medical intervention described herein.

In further embodiments, the method or use comprises administering the medical intervention to the patient. Appropriate methods of administering a medical intervention to a patient will be known to the person skilled in medicine.

Where a medical intervention has been selected and/or administered to a patient, it may be necessary to assess whether the medical intervention has been successful and accordingly whether the medical intervention should be discontinued or modified; or to assess whether the medical intervention has been unsuccessful and should be continued or modified. It will be understood that a medical intervention may be considered to be successful where the patient is no longer identified as predisposed to experiencing a severe episode of a disease by the presence of the biosignature comprising the biomarkers, as described herein, and/or identified as at risk of experiencing a severe episode of a disease by the presence of the biosignature comprising the biomarkers, as described herein, following the medical intervention. It will be understood that a medical intervention may be considered to be unsuccessful where the patient is identified as predisposed to experiencing a severe episode of a disease by the presence of biosignature comprising the biomarkers, as described herein, and/or identified as at risk of experiencing a severe episode of a disease by the presence of the biosignature comprising the biomarkers, as described herein, following the medical intervention.

Accordingly, in one aspect the disclosure provides a method of evaluating the effectiveness of a medical intervention in a patient, comprising the steps of: detecting the presence of a biosignature comprising the biomarkers hepatocyte growth factor (HGF) and Spondin-1 (SPON-1) in the patient's blood at a first time; applying the medical intervention to the patient; detecting the presence of the biosignature comprising the biomarkers hepatocyte growth factor (HGF) and Spondin-1 (SPON-1) in the patient's blood at a second time, and evaluating the effectiveness of the medical intervention by comparing the presence of the biosignature comprising three or more biomarkers, as described herein, in the patient's blood at a first time with the presence of the biosignature comprising three or more biomarkers, as described herein, in the patient's blood at a second time.

The detecting the biomarker or biomarkers, as described herein, at a first time may occur directly prior to the administration or application of the medical intervention, such as about 1 minute, about 2 minutes, about 3 minutes, about 4 minutes about 5 minutes, about 10 minutes, about 20 minutes, about 30 minutes, about 1 hour, about 2 hours, about 3 hours, about 4 hours, about 5 hours, about 6 hours, or more before the administration or application of the medical intervention. The detecting the biomarker or biomarkers, as described herein, at a first time may occur substantially before the administration or application of the medical intervention, such as about 12 hours, about 24 hours, about 2 days, about 3 days, about 4 days, about 5 days, about 6 days, about 1 week, about 2 weeks, about 3 weeks, about 4 weeks, about 1 month or more before the administration or application of the medical intervention.

The detecting the biomarker or biomarkers, as described herein, at a second time may occur directly after to the administration or application of the medical intervention, such as about 1 minute, about 2 minutes, about 3 minutes, about 4 minutes about 5 minutes, about 10 minutes, about 20 minutes, about 30 minutes, about 1 hour, about 2 hours, about 3 hours, about 4 hours, about 5 hours, about 6 hours, or more after the administration or application of the medical intervention. The detecting the biomarker or biomarkers, as described herein, at a second time may occur substantially after the administration or application of the medical intervention, such as about 12 hours, about 24 hours, about 2 days, about 3 days, about 4 days, about 5 days, about 6 days, about 1 week, about 2 weeks, about 3 weeks, about 4 weeks, about 1 month or more after the administration or application of the medical intervention.

Accordingly, depending on the duration of the medical intervention, the detecting the biomarker or biomarkers, as described herein, at a second time may occur about 2 minutes, about 4 minutes, about 6 minutes, about 8 minutes about 10 minutes, about 20 minutes, about 40 minutes, about 1 hour, about 2 hours, about 4 hours, about 6 hours, about 8 hours, about 10 hours, about 12 hours, about 24 hours, about 2 days, about 4 days, about 6 days, about 8 days, about 10 days, about 12 days, about 2 weeks, about 4 weeks, about 6 weeks, about 8 weeks, about 2 months or more after the detecting the biomarker or biomarkers, as described herein, at a first time occurs.

In one aspect the method of evaluating the effectiveness of a medical intervention in a patient comprises at least one step of providing a blood sample from a patient. Accordingly, in one aspect the disclosure provides a method of evaluating the effectiveness of a medical intervention in a patient, comprising the steps of: providing a first blood sample from the patient; detecting the presence of a biosignature comprising the biomarkers hepatocyte growth factor (HGF) and Spondin-1 (SPON-1) in the first blood sample; applying the medical intervention to the patient; providing a second blood sample from the patient; detecting the presence of a biosignature comprising the biomarkers hepatocyte growth factor (HGF) and Spondin-1 (SPON-1) in second blood sample from the patient, and evaluating the effectiveness of the medical intervention by comparing the presence of the biosignature comprising the biomarkers, as described herein, in the first patient's blood at a first time sample from the patient with the presence of the biosignature comprising the biomarkers, as described herein, in the patient's second blood sample.

As described herein, in some embodiments of any of the methods and uses provided herein, the biosignature further comprises at least one or more biomarkers selected from the group comprising or consisting of: TNF-related weak inducer of apoptosis (TWEAK); tissue factor pathway inhibitor (TFPI); pappalysin-1 (PAPPA); and CCL28. In some embodiments, the biosignature further comprises at least one or more biomarkers selected from the group comprising or consisting of: CXCL9, Azu-1, tryptase, myeloperoxidase (MPO), and/or neutrophil elastase.

Methods for providing a blood sample from a patient are described herein. The second blood sample is provided after the first blood sample has been provided, typically after the medical intervention has been administered or applied to the patient.

The first blood sample may be provided directly prior to the administration or application of the medical intervention, such as about 1 minute, about 2 minutes, about 3 minutes, about 4 minutes about 5 minutes, about 10 minutes, about 20 minutes, about 30 minutes, about 1 hour, about 2 hours, about 3 hours, about 4 hours, about 5 hours, about 6 hours, or more before the administration or application of the medical intervention. The first blood sample may be provided substantially before the administration or application of the medical intervention, such as about 12 hours, about 24 hours, about 2 days, about 3 days, about 4 days, about 5 days, about 6 days, about 1 week, about 2 weeks, about 3 weeks, about 4 weeks, about 1 month or more before the administration or application of the medical intervention.

The second blood sample may be provided directly after to the administration or application of the medical intervention, such as about 1 minute, about 2 minutes, about 3 minutes, about 4 minutes about 5 minutes, about 10 minutes, about 20 minutes, about 30 minutes, about 1 hour, about 2 hours, about 3 hours, about 4 hours, about 5 hours, about 6 hours, or more after the administration or application of the medical intervention. The second blood sample may be provided substantially after the administration or application of the medical intervention, such as about 12 hours, about 24 hours, about 2 days, about 3 days, about 4 days, about 5 days, about 6 days, about 1 week, about 2 weeks, about 3 weeks, about 4 weeks, about 1 month or more after the administration or application of the medical intervention.

Accordingly, depending on the duration of the medical intervention, the second blood sample may be provided about 2 minutes, about 4 minutes, about 6 minutes, about 8 minutes about 10 minutes, about 20 minutes, about 40 minutes, about 1 hour, about 2 hours, about 4 hours, about 6 hours, about 8 hours, about 10 hours, about 12 hours, about 24 hours, about 2 days, about 4 days, about 6 days, about 8 days, about 10 days, about 12 days, about 2 weeks, about 4 weeks, about 6 weeks, about 8 weeks, about 2 months or more after the first blood sample is provided.

Methods for detecting the presence of a biomarker or biomarkers, as described herein, are provided herein. Detecting the presence of a biosignature comprising the biomarkers hepatocyte growth factor (HGF) and Spondin-1 (SPON-1) in the patient's blood at a first time or in the first blood sample from the patient and/or in the patient's blood at a second time or in the second blood sample from the patient may comprise detecting the presence of a biosignature comprising biomarkers hepatocyte growth factor (HGF) and Spondin-1 (SPON-1) as provided herein. Administering or applying the medical intervention to the patient may comprise administering or applying the medical intervention to the patient as described herein.

Evaluating the effectiveness of the medical intervention requires comparing the presence of a biomarker or biomarkers, as described herein, in the patient's blood at a first time or in the first blood sample from the patient with the presence of the biomarker or biomarkers, as described herein, in the patient's blood at a second time or in the second blood sample from the patient may comprise comparing the level of said biomarker or biomarkers, as described herein, in the patient's blood at a first time or in the first blood sample from the patient with the level of said biomarker or biomarkers, as described herein, in the patient's blood at a second time or in the second blood sample from the patient. The level of the biomarker or biomarkers, as described herein, may be an absolute level or relative level of said biomarker or biomarkers, as described herein, such as an absolute mass or weight, concentration, or fold-change of said biomarker or biomarkers, as described herein. Absolute levels and relative levels of a biomarker or biomarkers, as described herein, and proteins are described herein. Methods of determining absolute levels and relative levels of a biomarker or biomarkers, as described herein, and proteins are described herein. The person skilled in the art of bioinformatics or statistics will readily understand methods of comparing the presence or level of a biomarker or biomarkers, as described herein, in the patient's blood at a first time or in the first blood sample from the patient with the level of said biomarker or biomarkers, as described herein, in the patient's blood at a second time or in the second blood sample from the patient.

A medical intervention may be evaluated as being effective or ineffective. To put another way, a medical intervention may be evaluated as being successful or unsuccessful.

In one embodiment, a medical intervention may be evaluated as being effective or successful if the presence or level of a biomarker or biomarkers, as described herein, in the patient's blood at a second time or in the second blood sample from the patient has not increased compared to the presence of the biomarker or biomarkers, as described herein, in the patient's blood at a first time or in the first blood sample from the patient. In other words, the presence of the biosignature has not changed. In one embodiment, a medical intervention may be evaluated as being effective or successful if the presence or level of a biomarker or biomarkers, as described herein, in the patient's blood at a second time or in the second blood sample from the patient has decreased compared to the presence or level of the biomarker or biomarkers, as described herein, in the patient's blood at a first time or in the first blood sample from the patient. In other words, the presence of the biosignature may be decreased; or the biosignature may be absent. A medical intervention may be evaluated as being effective or successful if the presence or level of a biomarker or biomarkers, as described herein, in the patient's blood at a second time or in the second blood sample from the patient has decreased by a defined fold-change compared to the presence or level of the biomarker or biomarkers, as described herein, in the patient's blood at a first time or in the first blood sample from the patient. The fold-change may be any fold-change as defined herein. A medical intervention may be evaluated as being effective or successful if the presence or level of a biomarker or biomarkers, as defined herein, in the patient's blood at a second time or in the second blood sample from the patient is below a threshold level, where the presence or level of the biomarker or biomarkers, as defined herein, in the patient's blood at a first time or in the first blood sample from the patient was above said threshold. The threshold may be any threshold as defined herein. Where the biomarker or biomarkers are below a threshold level, the biosignature may be absent.

A medical intervention may be evaluated as being ineffective or unsuccessful if the presence or level of a biomarker or biomarkers, as described herein, in the patient's blood at a second time or in the second blood sample from the patient is the same compared to the presence or level of the biomarker or biomarkers, as described herein, in the patient's blood at a first time or in the first blood sample from the patient. In other words, the presence of the biosignature has not changed. A medical intervention may be evaluated as being ineffective or unsuccessful if the presence or level of a biomarker or biomarkers, as described herein, in the patient's blood at a second time or in the second blood sample from the patient has not decreased compared to the presence or level of the biomarker or biomarkers, as described herein, in the patient's blood at a first time or in the first blood sample from the patient. A medical intervention may be evaluated as being ineffective or unsuccessful if the presence or level of a biomarker or biomarkers, as described herein, in the patient's blood at a second time or in the second blood sample from the patient has increased compared to the presence or level of the biomarker or biomarkers, as described herein, in the patient's blood at a first time or in the first blood sample from the patient. In other words, the presence of the biosignature has increased. A medical intervention may be evaluated as being ineffective or unsuccessful if the presence or level of a biomarker or biomarkers, as described herein, in the patient's blood at a second time or in the second blood sample from the patient has increased by a certain fold-change compared to the presence or level of the biomarker or biomarkers, as described herein, in the patient's blood at a first time or in the first blood sample from the patient. The fold-change may be any fold-change as defined herein. A medical intervention may be evaluated as being ineffective or unsuccessful if the presence or level of a biomarker or biomarkers, as described herein, in the patient's blood at a second time or in the second blood sample from the patient is above a threshold level, where the presence or level of the biomarker or biomarkers, as described herein, in the patient's blood at a first time or in the first blood sample from the patient was above said threshold. The threshold may be any threshold as defined herein.

Aspects of the disclosure provide a method of selecting a medical intervention for a patient, comprising the steps of: detecting the presence of a biosignature comprising the biomarkers (as described herein) as described herein; identifying that the patient is predisposed to experiencing a severe episode of a disease by the presence of a biomarker or biomarkers, as described herein, and/or identifying that the patient is at risk of experiencing a severe episode of a disease by the presence of the biosignature comprising the biomarkers, as described herein, and selecting a medical intervention for the patient.

In one aspect, method of selecting a medical intervention for a patient comprises at least one step of providing a blood sample from a patient. Accordingly, aspects of the disclosure provide a method of selecting a medical intervention for a patient, comprising the steps of: providing a blood sample from a patient; detecting the presence of a biomarker or biomarkers (as described herein) as described herein; identifying that the patient is predisposed to experiencing a severe episode of a disease by the presence of a biomarker or biomarkers, as described herein, and/or identifying that the patient is at risk of experiencing a severe episode of a disease by the presence of the biomarker or biomarkers, as described herein, and selecting a medical intervention for the patient.

Other aspects of the disclosure provide a method of preventing and/or treating a severe episode of a disease in a patient, comprising the steps of: detecting the presence of a biosignature comprising the biomarkers hepatocyte growth factor (HGF) and Spondin-1 (SPON-1) in the patient's blood; identifying that the patient is predisposed to experiencing a severe episode of a disease by the presence of the biosignature comprising the biomarkers and/or identifying that the patient is at risk of experiencing a severe episode of a disease by the presence of the biosignature comprising the biomarkers; selecting a medical intervention for the patient; and administering the medical intervention to the patient.

In some aspect, the method of preventing and/or treating a severe episode of a disease in a patient comprises at least one step of providing a blood sample from a patient. Accordingly, some aspects of the disclosure provide a method of preventing and/or treating a severe episode of a disease in a patient, comprising the steps of: providing a blood sample from the patient; detecting the presence of a biosignature comprising the biomarkers hepatocyte growth factor (HGF) and Spondin-1 (SPON-1) in the blood sample from the patient; identifying that the patient is predisposed to experiencing a severe episode of a disease by the presence of the biosignature comprising the biomarkers and/or identifying that the patient is at risk of experiencing a severe episode of a disease by the presence of the biosignature comprising the biomarkers; selecting a medical intervention for the patient; and administering the medical intervention to the patient.

Methods for providing a blood sample from a patient are described herein. Methods for detecting the presence of a biomarker or biomarkers, as described herein, are provided herein. Identifying that the patient is predisposed to experiencing a severe episode of a disease by the presence of a biomarker or biomarkers, as described herein, and/or identifying that the patient is at risk of experiencing a severe episode of a disease by the presence of the biomarker or biomarkers, as described herein, may comprise embodiments, instances, and examples as provided herein. Selecting a medical intervention for the patient may comprise selecting a medical intervention for the patient as described herein. Administering the medical intervention to the patient may comprise administering the medical intervention to the patient as described herein.

In addition to the methods and uses provided herein, the disclosure also provides a kit of parts for identifying if a patient is predisposed to experiencing a severe episode of a disease and/or at risk of experiencing a severe episode of a disease, comprising a means for detecting the presence of a biomarker or biomarkers comprised by the biosignature, as described herein.

In one embodiment, the biomarker is selected from the list consisting of: hepatocyte growth factor (HGF); TNF-related weak inducer of apoptosis (TWEAK); Spondin-1 (SPON-1); tissue factor pathway inhibitor (TFPI); pappalysin-1 (PAPPA); CCL28; CXCL9; azurocidin-1 (Azu-1) and C-reactive protein (CRP). In one embodiment, the biomarker is hepatocyte growth factor (HGF). In one embodiment, the biomarker is Spondin-1 (SPON-1). In one embodiment, the biomarker is tissue factor pathway inhibitor (TFPI). In one embodiment, pappalysin-1 (PAPPA). In one embodiment, the biomarker is CCL28. In one embodiment, the biomarker is CXCL9. In one embodiment, the biomarker is azurocidin-1 (Azu-1). In one embodiment, the biomarker is C-reactive protein (CRP).

In one embodiment, the biomarkers are three or more biomarkers selected from the list consisting of: hepatocyte growth factor (HGF); TNF-related weak inducer of apoptosis (TWEAK); Spondin-1 (SPON-1); tissue factor pathway inhibitor (TFPI); pappalysin-1 (PAPPA); CCL28; CXCL9; azurocidin-1 (Azu-1) and C-reactive protein (CRP). In one embodiment, the biomarkers are three or more biomarkers comprising hepatocyte growth factor (HGF) and two or more biomarkers selected from the list consisting of: TNF-related weak inducer of apoptosis (TWEAK); Spondin-1 (SPON-1); tissue factor pathway inhibitor (TFPI); pappalysin-1 (PAPPA); CCL28; CXCL9; azurocidin-1 (Azu-1) and C-reactive protein (CRP). In one embodiment, the biomarkers are three or more biomarkers comprising hepatocyte growth factor (HGF); TNF-related weak inducer of apoptosis (TWEAK); and one or more biomarker selected from the group comprising of: Spondin-1 (SPON-1); tissue factor pathway inhibitor (TFPI); pappalysin-1 (PAPPA); CCL28; CXCL9; azurocidin-1 (Azu-1) and C-reactive protein (CRP). In one embodiment, the biomarkers are three or more biomarkers comprising hepatocyte growth factor (HGF); TNF-related weak inducer of apoptosis (TWEAK); and Spondin-1 (SPON-1). In one embodiment, the biomarkers are three or more biomarkers comprising hepatocyte growth factor (HGF); TNF-related weak inducer of apoptosis (TWEAK); Spondin-1 (SPON-1); and biomarkers selected from the group consisting of: tissue factor pathway inhibitor (TFPI); pappalysin-1 (PAPPA); CCL28; CXCL9; azurocidin-1 (Azu-1) and C-reactive protein (CRP).

In a preferred embodiment, the biomarkers of the kit are all six of the biomarkers comprised by the biosignature and are selected from the group comprising or consisting of: hepatocyte growth factor (HGF); TNF-related weak inducer of apoptosis (TWEAK); Spondin-1 (SPON-1); tissue factor pathway inhibitor (TFPI); pappalysin-1 (PAPPA); and CCL28.

In a preferred embodiment, the biomarkers of the kit are all nine of the biomarkers selected from the group comprising or consisting of: hepatocyte growth factor (HGF); TNF-related weak inducer of apoptosis (TWEAK); Spondin-1 (SPON-1); tissue factor pathway inhibitor (TFPI); pappalysin-1 (PAPPA); CCL28; CXCL9; azurocidin-1 (Azu-1) and C-reactive protein (CRP).

In one embodiment, the kit of parts comprises components that are suitable for the collection of a blood sample from a patient. Such components may be selected from the group comprising or consisting of: needles, syringes, cannulas, catheters, collection tubes (e.g., vacutainer blood collection tubes), blood bottles, or any combination thereof.

In one embodiment, the kit of parts comprises components that are suitable for sample processing. For example, the kit of parts may comprise components that are suitable for use in centrifuging blood samples, separating components of blood samples, or storing blood samples. Such components may be selected from the group comprising or consisting of: centrifuge tubes, Eppendorf tubes, pipettes, pipette tips, stripettes, chemicals such as heparin, or EDTA (solid or in solution), cryotubes, or any combination thereof.

In one embodiment, the kit of parts comprises components that are suitable for conducting protein assays. For example, the kit of parts may comprise components suitable for use in Proximity Extension Assays (PEAs) or ELISA. Such components may be selected from the group comprising or consisting: protein components, including primary antibody, secondary antibody, DNA polymerase, RNA polymerase, or any combination thereof; chemical entities, including primers, NTPs, dNTPs, ions, cations, salts, acids, radioisotopes, or enzyme substrates (e.g., ABTS or TMB), or any combination thereof; and/or structural components, such as multi-well plates (e.g., 96-well plates, 192-well plates, 384-well plates), centrifuge tubes, Eppendorf tubes, pipette tips, stripettes, or any combination thereof; or any combination thereof. The protein components of the kit of parts may be modified. Suitable modifications include, for example, oligonucleotide conjugates (e.g., DNA conjugates), protein conjugates (e.g., horseradish peroxidase conjugates and fluorescent protein conjugates), and chemical entity conjugates (e.g., radioisotope conjugates or fluorescent moiety conjugates).

In one embodiment, the kit of parts comprises instructions, for example an instruction leaflet or booklet. The instruction leaflet or booklet may comprise suitable instructions for conducing the method or use according to any of the aspects of the present disclosure.

The kit of parts may be suitable for use in the methods or uses according to any of the aspects of the disclosure provided herein.

The disclosure additionally provides methods, uses, and kits substantially as described herein.

### EXAMPLES

The following Examples provide non-limiting and exemplary methods for practicing aspects and embodiments of the invention. It is understood that variations, improvements, and equivalents of the methods, aspects, and embodiments described hereinbelow will occur to the person skilled in the art. Such variations, improvements, and equivalents are contemplated by the present disclosure and fall within the scope of the matter disclosed and claimed herein.

### Example 1

### Introduction

Coronary artery disease remains a major cause of death and disability worldwide. This is despite preventative measures such as cessation of smoking and the beneficial use of lipid-lowering treatments and blood pressure control^{1,2}. Therefore, much is still to be understood about the mechanisms underlying residual cardiovascular risk. Genetic studies have revealed new potential insights suggesting that altered levels of certain proteins, lipids, and metabolites may identify patients at higher risk³⁻⁶. Additionally, several studies have identified individual circulating biomarkers associated with risk of major adverse coronary events (MACE), principally by measuring biomolecule levels in systemic blood samples and often combined with coronary artery imaging to measure disease severity⁷⁻¹³. Symptoms of coronary artery disease can be relieved with percutaneous catheter-based coronary interventions, but accurate prediction of their risk of future events is not currently possible¹⁴.

It would be ideal to be able to stratify both asymptomatic people and symptomatic patients so that their medical management is precisely linked to their true risk of MACE. Imaging techniques such as electron beam computed tomography (EBCT) have been used to measure coronary calcification, a highly specific indicator of atherosclerotic disease that is a powerful predictor of myocardial infarction, vascular complications and mortality^{15,16}. However, calcification measurement is not used in routine clinical practice. Other imaging approaches include assessment of coronary inflammation to aid risk prediction¹⁷. Inflammation is thought to contribute to the destabilisation of atherosclerotic plaques, however, the causal components of the inflammatory pathways are only beginning to be understood^{18,19}. Assessment of general systemic inflammation by measuring C-reactive protein (CRP) levels (a non-specific acute phase reactant) has been linked with increased risk of myocardial infarction²⁰, and may be useful in stratifying patients most likely to benefit from targeted anti-inflammatory treatments²¹.

Part of the difficulty in identifying circulating biomarkers that are coronary in origin is that routine systemic blood sampling reflects coronary-released factors in vastly diluted quantities and systemic blood contains biomolecules from sources other than the coronary arteries, which may hinder efforts to identify unique coronary disease-derived factors. Some studies have addressed this by sampling tissue at the site of disease using proteomic approaches in ex vivo atherosclerotic plaques^{22,23} or coronary thrombi²⁴, while others have used aspiration or guide-wire catheters to sample blood directly from the coronary artery^{25,26}. Other approaches involve collection of blood from the coronary sinus, a venous reservoir reflecting whole coronary circulation where some subtle elevations in proteins were found in comparison with the systemic circulation, possibly reflecting coronary disease status more accurately than analysis of systemic samples alone²⁷.

In the present study, we used a novel device designed to collect multiple blood samples concurrently from the coronary artery, the PlaqueTec Liquid Biopsy System (LBS). The LBS has the unique capability of identifying local gradients of biomolecules by collecting blood samples simultaneously from coronary arteries at precise locations proximal and distal to a specific atherosclerotic plaque. The LBS also has structures that ensure efficient collection of biomolecules from the slower moving blood flow close to the artery wall. Our previous studies in a small cohort named Clinical Study 1 (CS1) identified several gradients in biomolecules across plaques²⁸. Here we investigated absolute levels of proteins and report a set of proteins that was discovered at higher levels in coronary blood samples for the majority of patients, compared with systemic blood samples. Further, this protein set was also observed at elevated levels in the periphery of a minority of patients, but not in healthy controls. We aimed to establish if the systemic biosignature was present in other cohorts and if it could be useful in reflecting a particular coronary status.

### Materials and Methods

### Coronary Artery Blood Sample Collection, CS1 Cohort

12 patients (45-74 years, all male), underwent blood sampling prior to balloon dilatation of an obstructive coronary artery plaque as part of a scheduled percutaneous coronary intervention (PCI) procedure. Samples were obtained simultaneously via sampling ports positioned either side of an identified plaque, using the LBS as described previously²⁸. All patients received dual anti-platelet therapy and periprocedural anticoagulation with heparin. A systemic blood sample was taken prior to heparin administration, and another systemic blood sample was taken after the PCI procedure. Study protocols were approved by the local ethics committee (NRES London—Chelsea 13/LO/0954) and the institutional review board at Papworth Hospital, Cambridge, U.K. Participants gave written, informed consent. LBS-derived and systemic samples were mixed with EDTA at a final concentration of 1.75 mg/ml and plasma isolated by centrifugation at 10,000×g to generate platelet-poor plasma and stored at −80° C. until analysis.

### Healthy Control Plasma

Control plasma from 12 healthy individuals was obtained from a commercial source (BioIVT) for comparison with the CS1 cohort. The selected individuals had similar features to the CS1 population (all male, age range 46-72).

### PACIFIC Cohort Data

As detailed in a study by Bom et al (2019)⁹, systemic blood samples were collected in EDTA from patients after fasting for at least 12 hours. Data from 196 patients in the PACIFIC cohort was accessed via the journal website⁹. Data from selected proteins were plotted as scatter plots in Excel for comparison with CS1 data.

### Healthy Control Cohort (SCAPIS)

For comparison to further healthy controls, data available in graph form was accessed (www.proteinatlas.org/blood). This was part of a study detailing the human secretome involving 86 healthy individuals (age 50-65 years) from the Swedish Cardio Pulmonary bioImage Study (SCAPIS). The study measured 748 proteins in plasma by PEA (Olink) over a 1-year period with 4 consecutive visits with 3 month intervals.

### Healthy Control Cohort (Olink)

Data on normal ranges of proteins measured by PEA for 300 healthy individuals was accessed at Olink (https://insight.olink.com/data-stories/normal-ranges). Data was in the form of interquartile ranges (IQR), with outliers included in the calculations.

### Respiratory Disease/COVID-19 Cohort

384 patients enrolled in the emergency department during the COVID-19 peak between March and April 2020 from Boston MGH had blood plasma samples analysed on the Olink Explore 1536 protein panel²⁹. Protein data and patient outcomes for this study were accessed via the Olink website (Olink.com).

### ESKD/COVID-19 Cohorts

Data was accessed from a study of end-stage kidney disease (ESKD) subcohort A (plasma) where 55 patients tested positive and 51 tested negative for COVID-19, and from subcohort B (serum) where 46 patients tested positive and 11 tested negative for COVID-1930. Protein level data and patient outcomes for this study were accessed via the journal website.

### Protein Assays

### Proximity Extension Assay (PEA)

CS1 plasma samples were analysed by PEA at Olink, Uppsala, Sweden, initially using the CVD1 panel (currently discontinued). Assay characteristics with detection limits and validations are available from the manufacturer's webpage (http://www.olink.com/products/proseek-multiplex/downloads/data-packages). Separate analysis was performed on CS1 plasma samples using the CVD3 and Inflammation panels, each containing 92 proteins. 83 proteins from the CVD1 panel, 92 from the CVD3 panel and 77 from the Inflammation panel passed strict quality controls and results presented in NPX units. Healthy control samples from BioIVT were analysed together with CS1 samples on the CVD3 and Inflammation panels so that direct comparisons could be made.

### ELISA

CRP levels for CS1 were analysed by high sensitivity CRP (hsCRP) ELISA (Invitron, cat #IV3-105E) at Eurofins and results provided in mg/L.

### Statistical Analysis

Calculations of means, medians, outliers, centiles and use of conditional formatting to identify patient protein patterns was performed in Excel. To test for statistical significance between proteins levels in plasma sampled at different locations and timing (systemic v coronary, and pre- and post-PCI), a one-way ANOVA mixed-effects model, followed by a pairwise Holm-Sidak's multiple comparison's test was used (with an alpha of 0.05). These options were selected due to 12 patients having samples pre-PCI, while 11 of these patients had post-PCI samples collected. Statistical tests were performed in GraphPad (version 9.0). Graphs were created in either Excel of GraphPad. For the respiratory disease COVID-19 cohort survival analysis, Kaplan-Meier curves and statistics were created in GraphPad (version 9.0).

### Results

### Identification of Proteins at Higher Levels in Coronary v Systemic Blood Samples

Using the LBS, samples were obtained from 12 patients undergoing balloon dilatation of an obstructive coronary artery plaque as part of a scheduled PCI procedure in the CS1 trial. Initially, one panel of 92 proteins was analysed, CVD1 panel (Olink, currently unavailable). Using this panel, levels of hepatocyte growth factor (HGF) and pappalysin-1 (PAPPA) were identified at much higher levels (>10-fold) in the coronary samples derived from the upstream port (FIG. 1A) or downstream port (FIG. 1B), compared with systemic samples for 10/12 individuals prior to the PCI procedure. Spondin-1 (SPON1) had the same patient pattern as HGF and PAPPA but to a lesser extent (FIG. 1A).

For the 2 patients that did not display a high coronary: systemic ratio, this was not due to low coronary levels but to high systemic signals for HGF, PAPPA and SPON1 (FIGS. 1C, D and E). Additionally, in post-PCI samples, the majority of patients had higher levels of HGF, PAPPA and SPON1 systemically. Some other proteins had higher levels post-PCI for the same patients, compared with pre-PCI, but to a lesser extent or in fewer patients (e.g., growth hormone) (FIG. 1F). These results indicated that the conditions of the PCI procedure had caused elevations in levels of these proteins. This could be caused by either (1) the presence of heparin, known to affect the concentrations of several proteins in blood, or (2) the procedure itself, introducing some damage or inflammation to the coronary artery, or a combination of both heparin and damage, and/or (3) the timing between sampling pre- and post-PCI. However, the intriguing pattern of high levels of HGF, PAPPA and SPON1 observed in systemic samples for 2 patients at baseline, prior to heparin administration and prior to the procedure prompted further investigation.

Analysis of CS1 Plasma in CVD3 and Inflammation Protein Panels and Comparison with Healthy Plasma

As a first step in validating the results, a repeat experiment was performed in the same samples from the CS1 cohort using the ‘Inflammation’ and ‘CVD3’ panels (Olink), where there is considerable overlap in proteins from the original CVD1 panel (discontinued). HGF is one of the proteins currently on the Inflammation panel and SPON1 on the CVD3 panel. PAPPA was not present on these panels. Plasma samples from 12 healthy individuals (age- and sex-matched) were included in these assays for comparison.

HGF levels displayed similar patterns when measured on 2 different occasions in 2 different panels (FIGS. 2A and B). The 10 patients with low HGF systemically prior to PCI had similar levels to healthy controls, indicating that the 2 patients with elevated HGF had systemic levels well above ‘normal’. Analysis of coronary/systemic protein ratios from the Inflammation panel revealed 3 further proteins with distinctly higher levels in the coronary plasma compared with systemic plasma; monokine induced by interferon γ (MIG, or CXCL9), C—C motif chemokine ligand 28 (CCL28) and TNF-related weak inducer of apoptosis, TNFSF12 (or TWEAK) (FIG. 2C). When analysing the CVD3 panel, SPON1, tissue factor pathway inhibitor (TFPI) and azurocidin-1 (Azu-1) had distinctly higher coronary:systemic ratios (FIG. 2D). On this repeat analysis using the Inflammation panel, SPON1 coronary and systemic levels differences were more pronounced, compared with results obtained using the CVD1 panel. Differences between NPX values measured on CVD1 and CVD3/Inflammation panels are most likely due to the different dilutions and dynamic ranges of the newer panel assays.

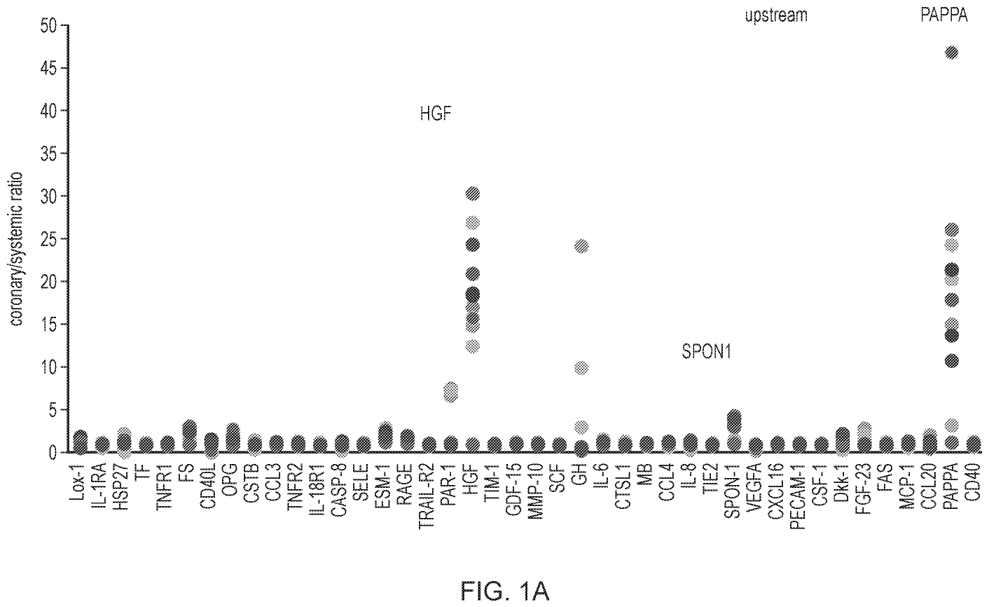

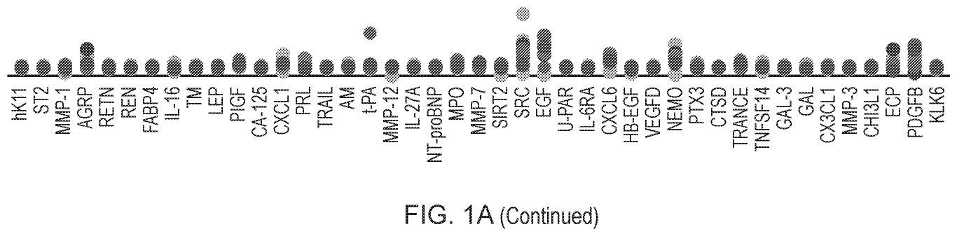

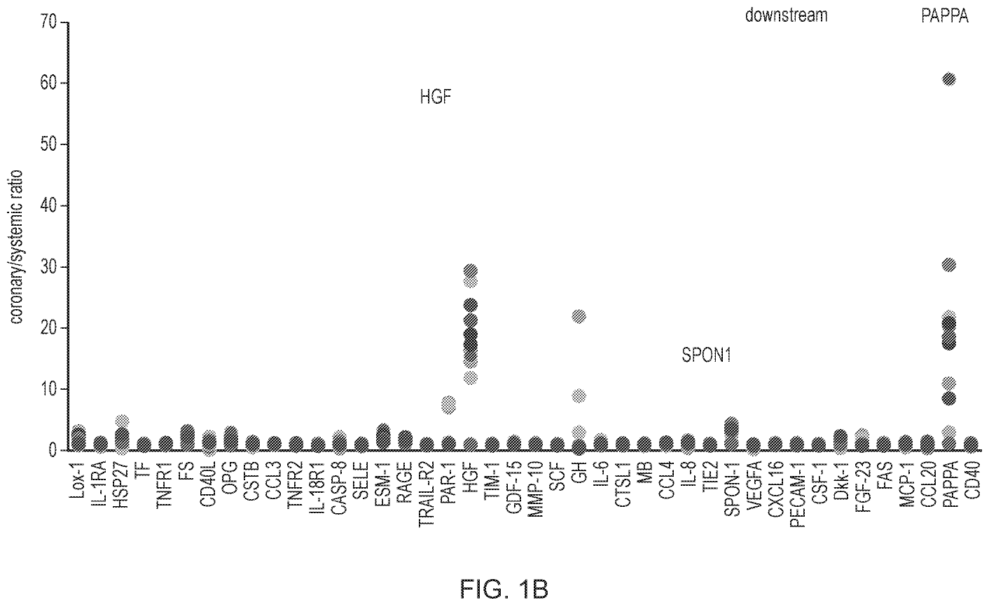

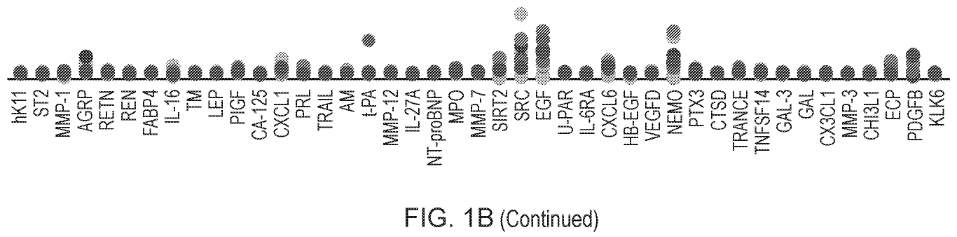

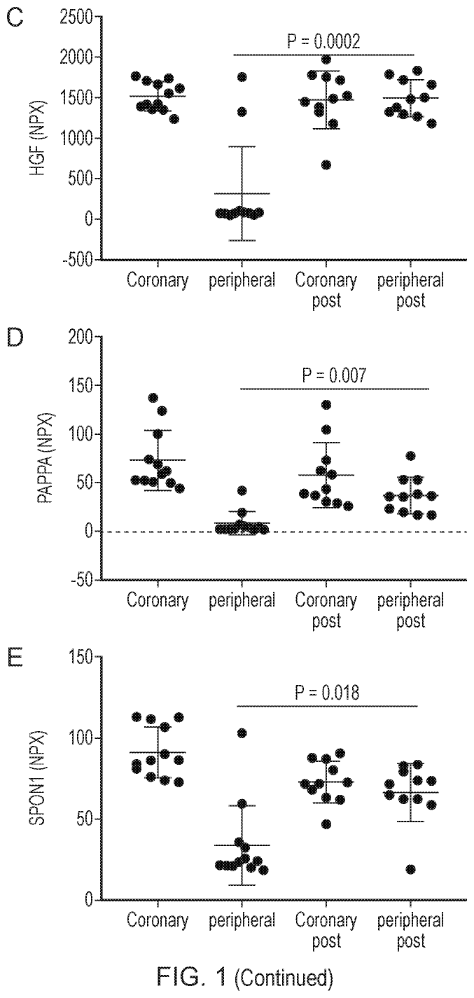

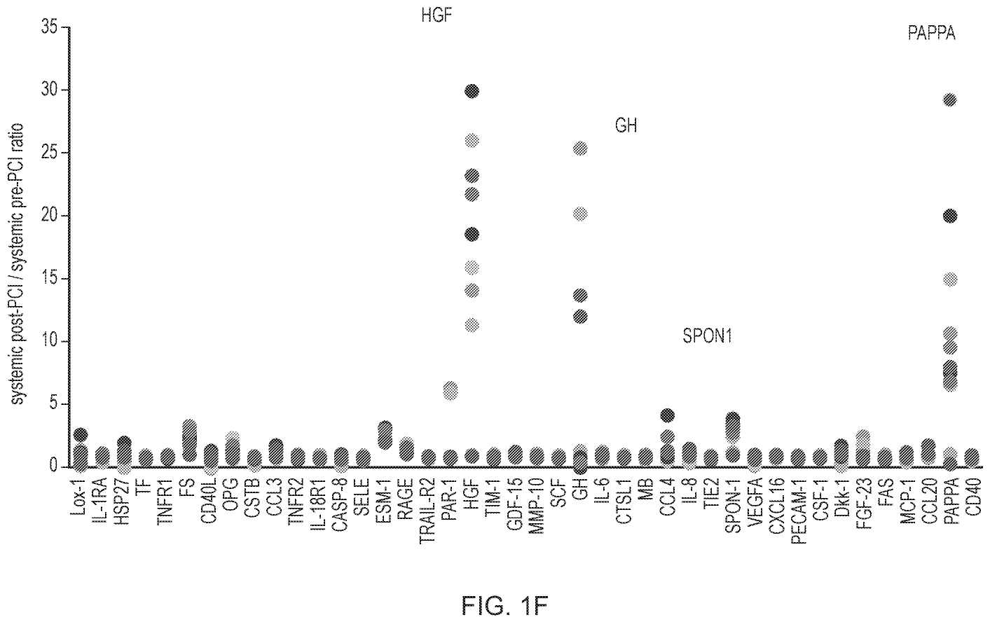

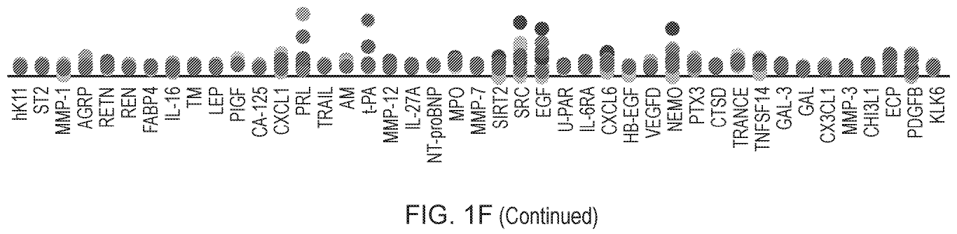

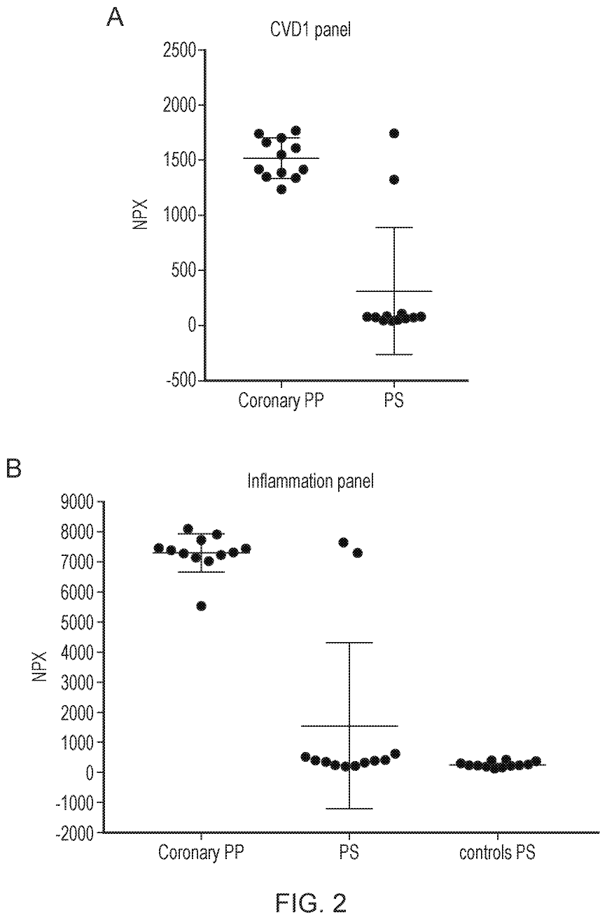

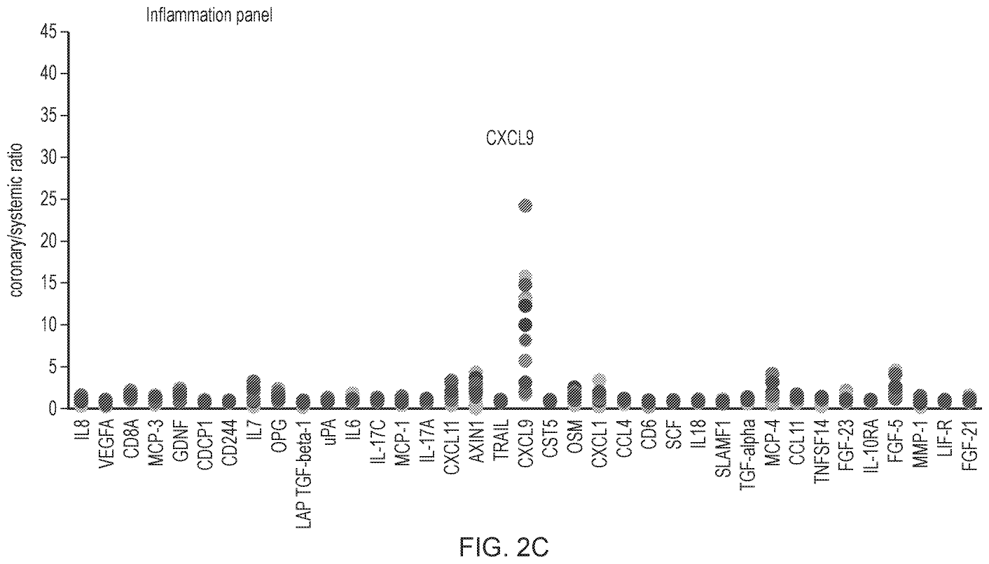

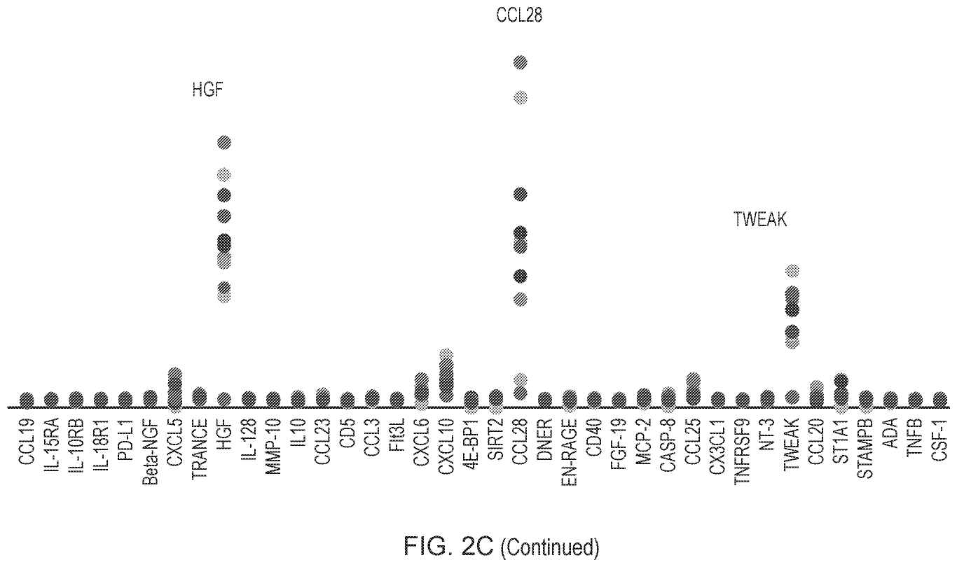

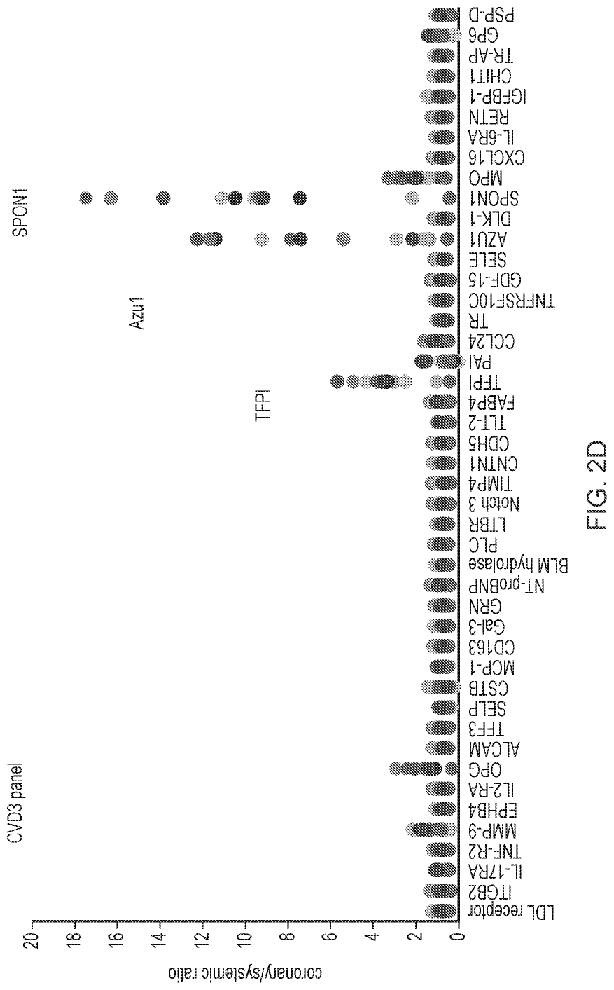

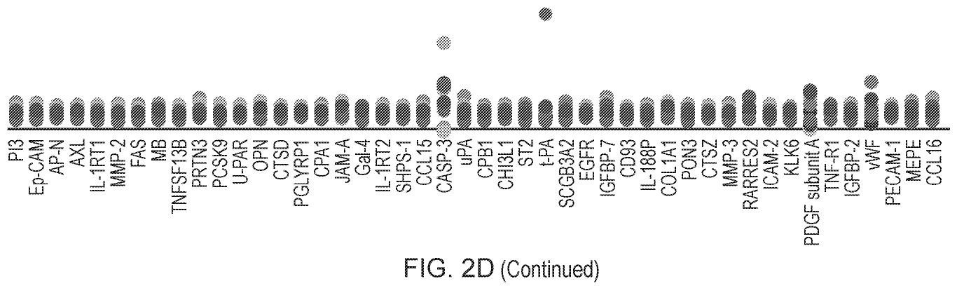

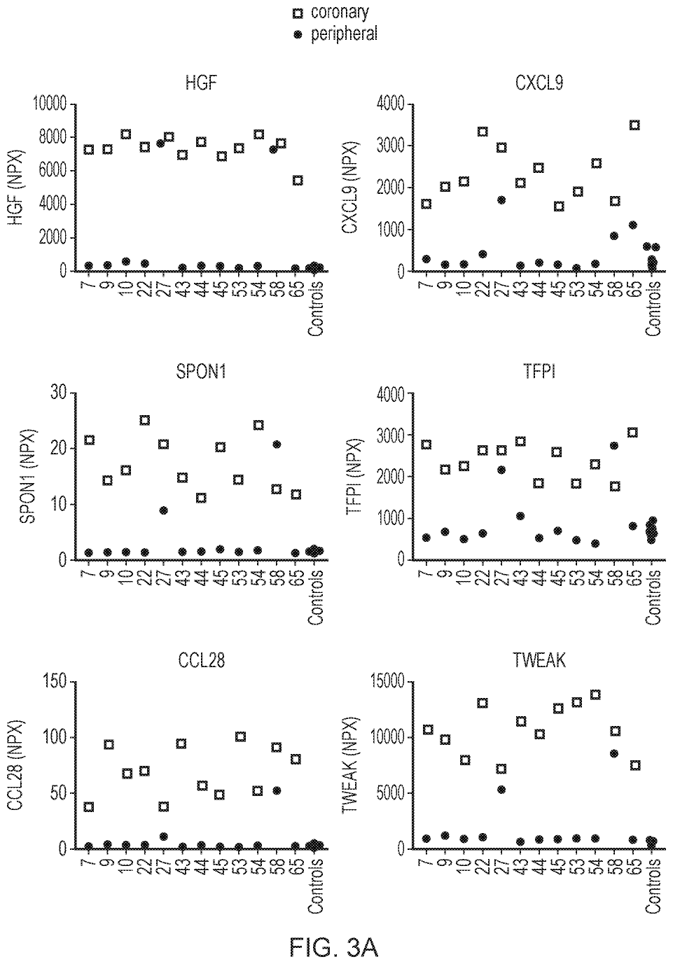

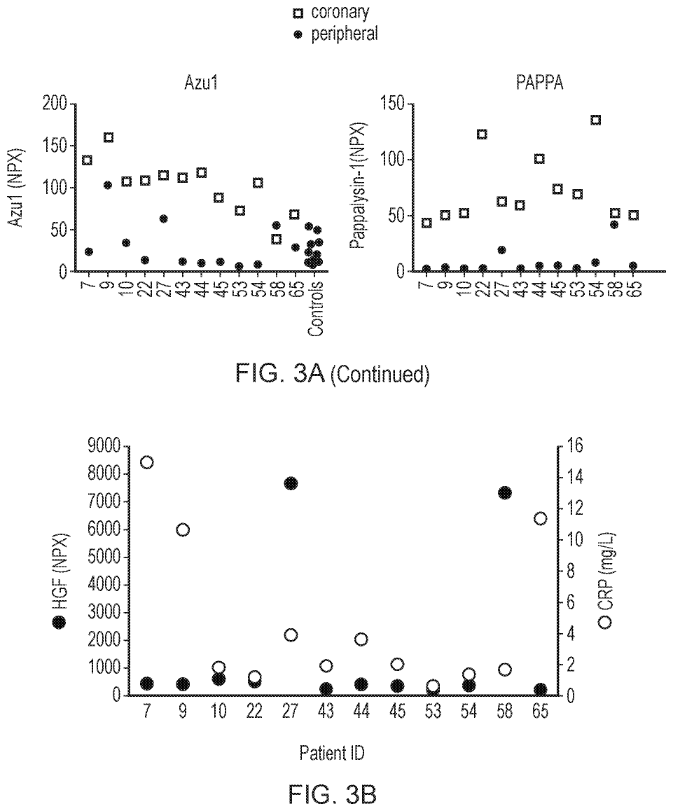

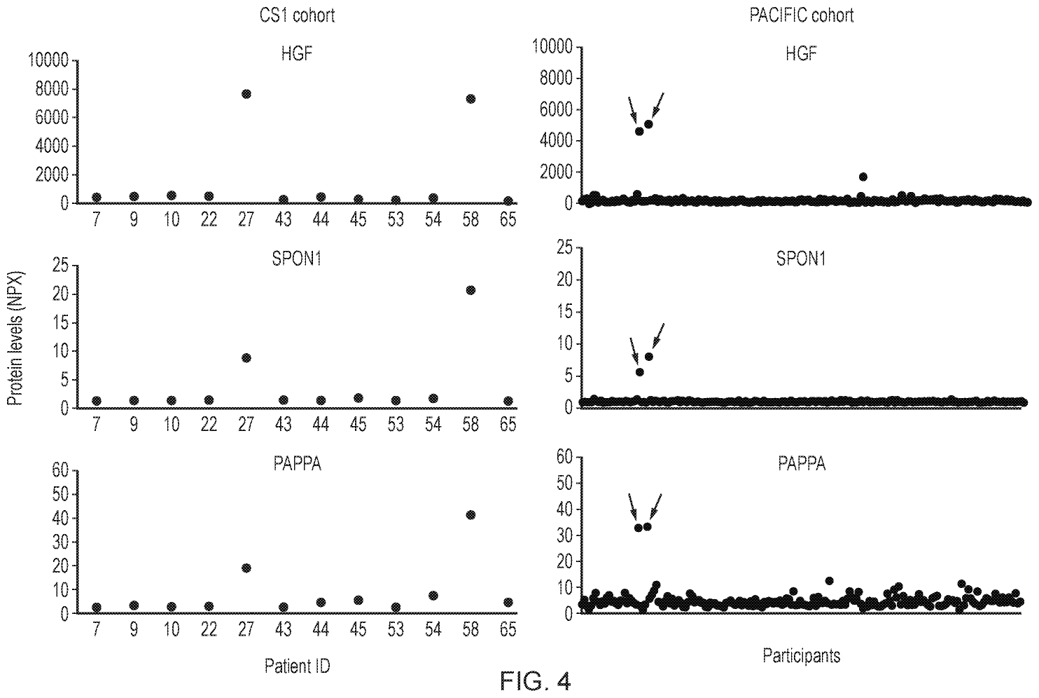

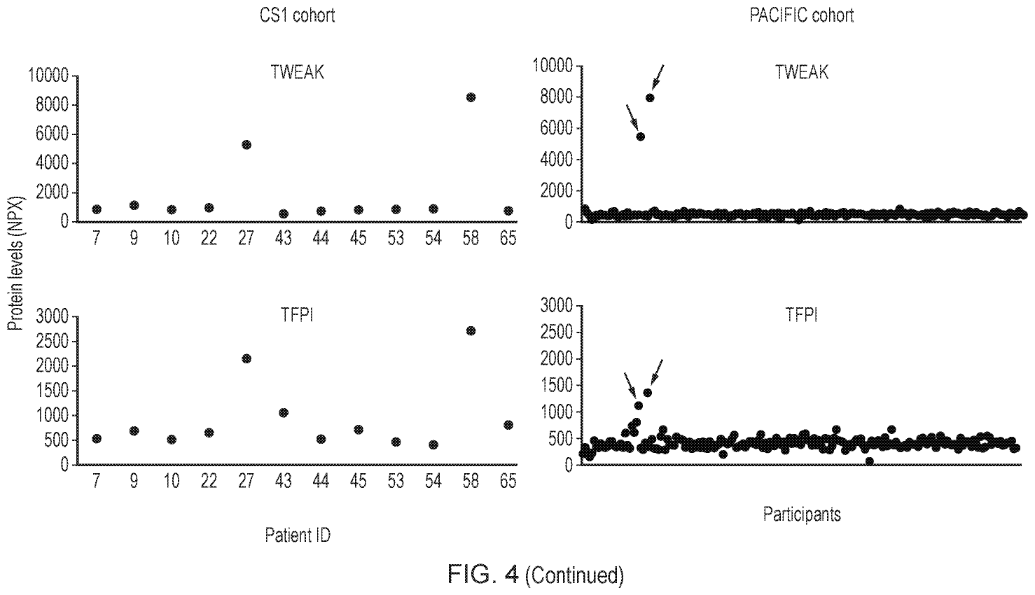

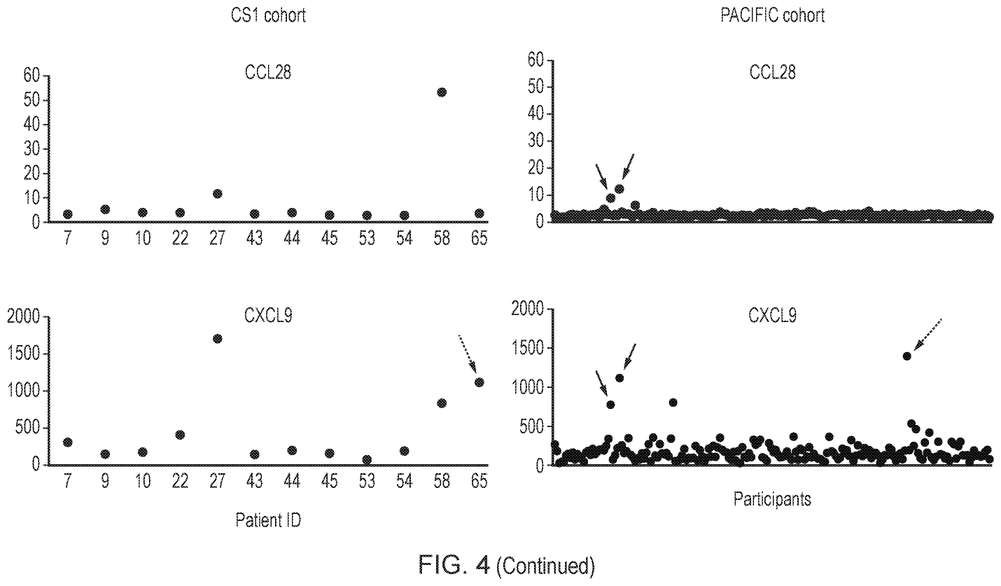

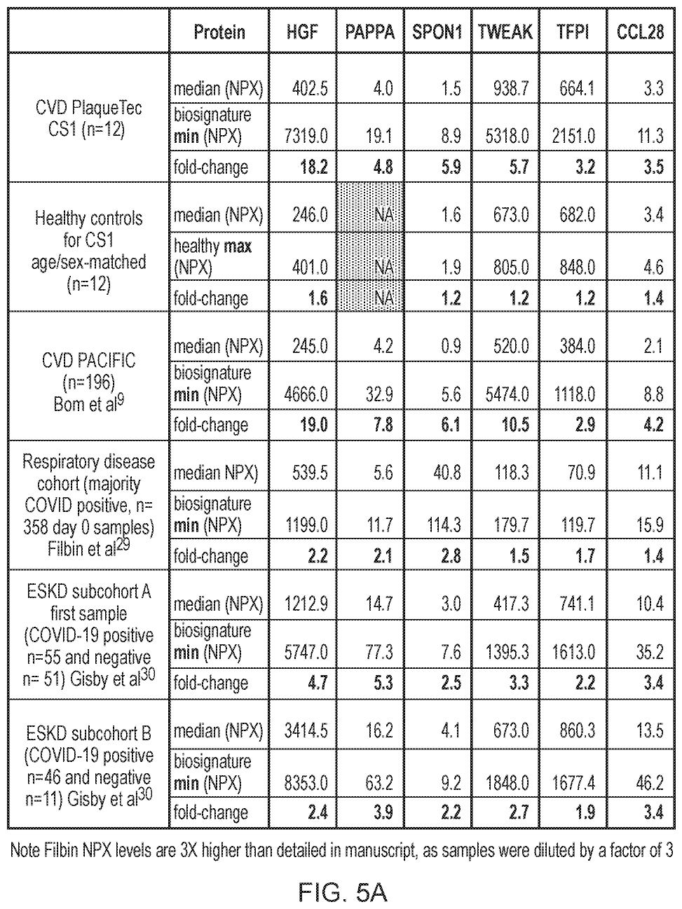

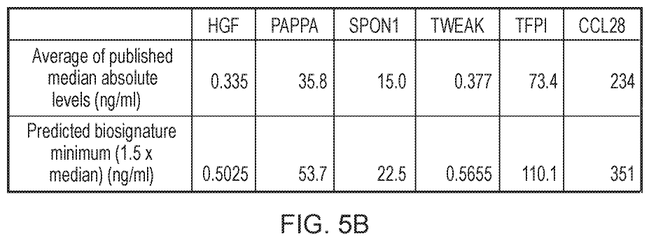

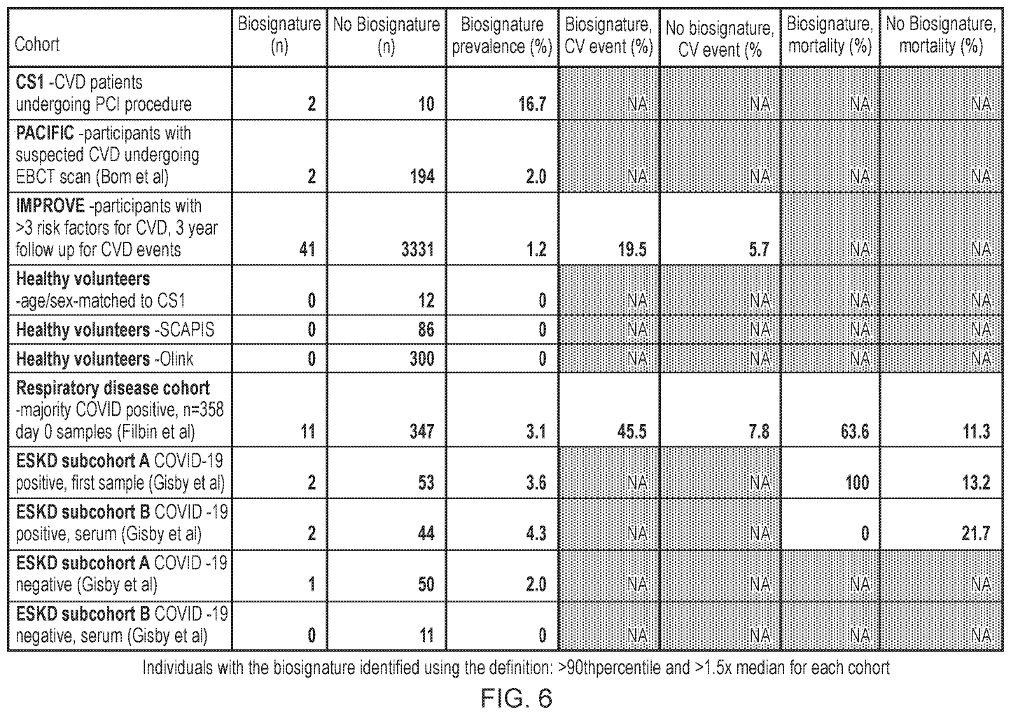

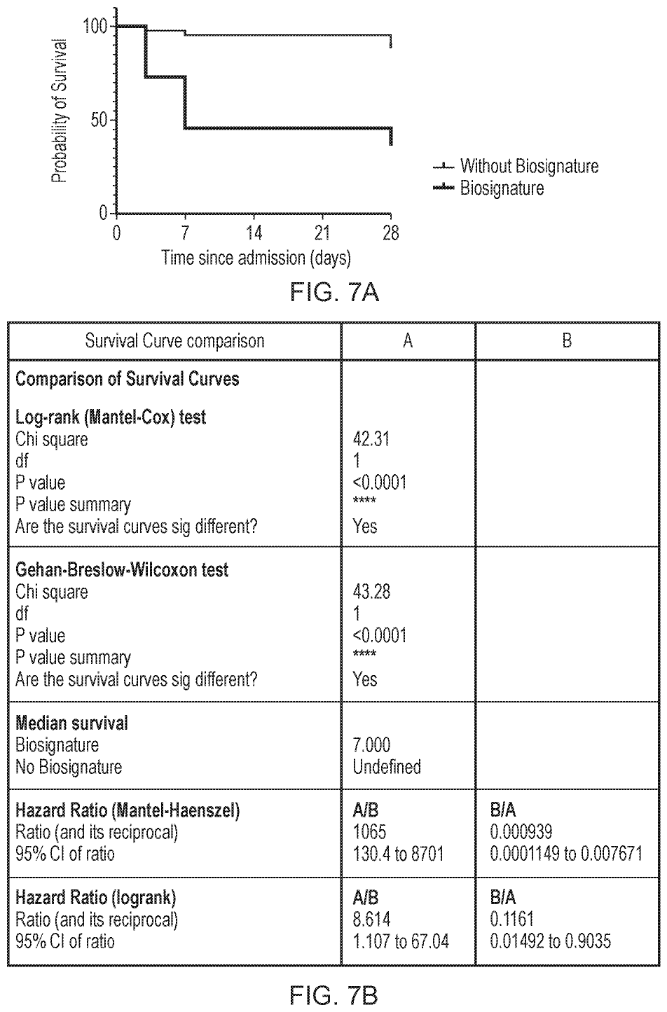

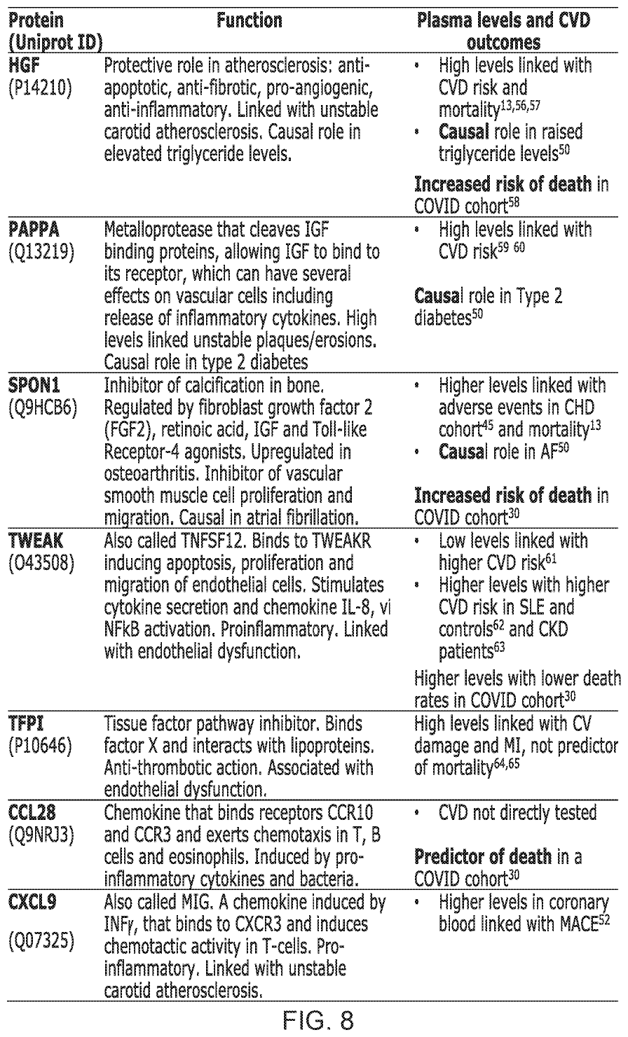

The proteins displaying raised levels in coronary plasma were then plotted as absolute values (FIG. 3A). A distinct pattern was seen for HGF, and the other 7 proteins followed a similar pattern to different extents, with Azu-1 displaying the least consistent pattern with one patient having higher systemic than coronary Azu-1. Healthy control levels for this set of proteins were generally in line with the CS1 systemic samples that exhibited low levels, with the exception of CXCL9 having 2 apparently healthy controls with above average levels, and Azu-1, with a relatively wide range of values (from 1-50 NPX units). Further, Azu-1 was only found at higher levels in the periphery for one of the 2 patients that displayed the ‘biosignature’ proteins. Therefore, Azu-1 and CXCL9 were considered less important in identifying individuals with the systemic biosignature pattern.

### Analysis of CRP Levels

To investigate if the 2 patients displaying the systemic biosignature pattern could be explained by general systemic inflammation, CRP levels were measured in a high-sensitivity assay (hsCRP) and found to be mid/low (<5 mg/L), and CRP did not correlate with HGF levels (FIG. 3B). Therefore, acute phase systemic inflammation did not appear to account for the high systemic protein levels observed in patients with the systemic biosignature.

### Investigating the Systemic Biosignature Pattern in Different Cohorts—PACIFIC

To find out if similar protein patterns were observed in patients from other cohorts, data from a study by Bom et al was accessed⁹. This study included a subset of patients from the PACIFIC cohort with suspected coronary artery disease. Using this data, the protein patterns for the selected biosignature proteins identified in CS1 were compared with the PACIFIC cohort (FIG. 4). The PACIFIC cohort appeared to have 2 patients with the biosignature (from a total of 196 patients). While several patients had well-above average levels for specific proteins, only 2 patients consistently had raised levels for biosignature proteins and did not have raised CRP levels (hsCRP <2.5 mg/L). The biosignature had therefore been validated in the PACIFIC cohort. With the available information on calcification scores from the PACIFIC cohort, for the 2 patients displaying the systemic biosignature, neither had a score of 0, nor a very high score indicating both individuals had low/mid-level atherosclerosis.

### Healthy Control Cohort (SCAPIS)

In addition to the 12 healthy control plasma samples analysed, for comparison to further healthy controls, data available as an open-access resource was accessed (www.proteinatlas.org/blood)³¹. Although raw data was not available, data presented on graphs revealed that some of the healthy subjects had variable levels of proteins over the 1-year period, suggesting that fluctuating levels of proteins may be regarded as ‘normal’. Of note with specific reference to the systemic biosignature, protein levels that varied widely in a minority of subjects included SPON1, HGF, TWEAK and TFPI, but there was no evidence for an elevated set of the biosignature proteins at a particular timepoint in this healthy cohort (summary of results in FIG. 6).

### Olink Healthy Cohort

Using data on variability (IQR) of proteins in a healthy cohort of 300 individuals, none of the biosignature proteins had high IQR values, suggesting it was unlikely that any of the participants in this cohort had the biosignature pattern (https://insight.olink.com/data-stories/normal-ranges, summary of results in FIG. 6).

### Biosignature

The biosignature was identified as a minority of patients with cardiovascular disease displaying distinctly higher levels of specific proteins, compared with the majority of the cohort investigated. In an attempt to define the biosignature, so that it can be identified in other cohorts, the fold-changes in protein levels from the median level was investigated (FIG. 5).

### Respiratory Disease/COVID-19 Cohort

A biosignature definition that aided identification of participants with the biosignature and applied to all cohorts was: >90^{th} percentile and >1.5× median values for HGF, PAPPA and SPON1. Using this definition, analysis of data accessed via Olink (Olink.com) from a study by Filbin et al²⁹ of respiratory disease/COVID-19 cohort at day 0 identified 11/358 patients with patterns resembling the higher levels of the biosignature proteins seen in CS1 and PACIFIC cohorts (FIG. 6). Individuals with the biosignature had a higher number of cardiac events within the first 72 hours of presentation (5/11), compared with those without the biosignature (29/373). In this cohort, a total of 49/384 patients died within 28 days of admission to hospital (12.7% mortality). For individuals identified with the biosignature using day 0 plasma protein levels, there was a striking difference in patient mortality: 7 of the 11 patients with the biosignature died within 28 days of admission to hospital (63.6%), while 43 of the 373 patients without the biosignature died (11.3%). Survival analysis indicated a highly significant statistical difference in probability of survival between those with and without the biosignature (P<0.001, FIG. 7), however confidence intervals are large. CRP levels were similar between the biosignature and non-biosignature groups.

### ESKD COVID-19 Cohort

The same biosignature definition of >90^{th} percentile and >1.5× median values for HGF, PAPPA and SPON1, was applied to protein level data for 2 end-stage kidney disease (ESKD) cohorts: subcohort A and B³⁰. Of the patients that tested positive for COVID-19, 2/55 patients in subcohort A and 2/51 patients in subcohort B were identified with the biosignature (FIG. 6). In subcohort A, both patients with the biosignature died, while in subcohort B both patients survived and were classed as having ‘severe’ and ‘moderate’ disease. In subcohort A the biosignature group had higher CRP (mean 220 mg/L) than the remaining cohort (mean 75 mg/L). Comparisons between the groups are summarised in FIG. 6.

### Discussion

Here, we present a systemic biosignature that was discovered serendipitously by investigating coronary and systemic levels of proteins in a small cohort of patients with coronary artery disease. There was a distinct discrepancy between the absolute levels of certain proteins in the diseased coronary artery compared with the periphery. The level of the proteins within the periphery increased after the PCI procedure, and this was most pronounced in the case of HGF. The PCI procedure can cause some damage and inflammation within the coronary artery as well as the intended disruption of the target atherosclerotic plaque. However, with the number of variables such as the presence of anticoagulant and damage that exist during a PCI procedure even prior to balloon angioplasty or stent implantation, any conclusions from the coronary comparison with systemic protein levels alone are speculative. The use of heparin cannot be avoided in PCI procedures, and its effects on rapidly altering concentrations of proteins in blood samples have been well documented^{32,33}, particularly in the release of TFPI which contributes to heparin's anti-coagulant effects³⁴ and selective release of HGF from the endothelial glycocalyx³⁵. Of the many blood protein levels altered by heparin, these were reported by Ngo et al, to include HGF, TWEAK, TFPI, CCL28 and SPON1, while PAPPA was not affected by heparin and CXCL9 was not included in the study³². However, other studies have reported heparin interactions with PAPPA³⁶ and CXCL⁹³⁷. It therefore appears that the biosignature proteins are among the many proteins that may be affected by heparin, although some proteins known to be heparin-sensitive were unchanged in the coronaries compared with systemic levels (e.g., heparin-binding epidermal growth factor (HBEGF), vascular endothelial growth factor (VEGF-A/D) and leptin. With the currently available data, it is likely that heparin has a role in inducing higher levels of proteins found in the coronary circulation in our study, compared with the periphery, although the presence of heparin does not fully explain the pattern we observed.

The finding of the biosignature in plasma at baseline (in the absence of heparin) in a minority of patients by systemic blood sampling prompted investigation in other cohorts. Indeed, the biosignature was found in a minority of patients from a cohort of individuals with suspected coronary disease (PACIFIC cohort)⁹. The biosignature was not found in healthy age-matched control plasma from a commercial source and appeared to be absent in healthy cohorts. The systemic biosignature may therefore be related to coronary disease and prompted further investigation.

The known functions of the biosignature proteins are summarised in FIG. 8. With all having direct or indirect roles in inflammation and endothelial function, the biosignature could possibly indicate active endothelial damage³⁸⁻⁴¹. Some, but not all of the biosignature proteins have previously been linked with risk of adverse cardiovascular events. For example, TWEAK plays a role in inflammation and repair in several diseases and is thought to promote the development of atherosclerosis, but lower circulating levels were reported in association with higher cardiovascular risk⁴². Higher levels of TFPI have been linked with cardiovascular damage and myocardial infarction, but is not a predictor of mortality^{43,44}

More recently, elevation in SPON1 was detected prior to cardiovascular events in CHF patients⁴⁵. SPON1 and CCL28 elevated levels were linked with increased risk of death in a CKD and COVID-19 cohort, and interestingly increased levels of TWEAK were associated with reduced risk of death in this cohort³⁰. Elevated HGF levels have been linked with worse outcomes in dialysis patients⁴⁶ and also more recently in COVID-19 cohorts⁴⁷⁻⁴⁹ with neutrophil activation suggested as a possible source of the high HGF levels preceding critical illness⁴⁹. In a recent genome-wide meta-analysis of 85 proteins in over 30,000 individuals (SCALLOP study) several proteins were identified as causative in disease and these included HGF (linked with high triglyceride levels), SPON-1 (linked with atrial fibrillation) and PAPPA (linked with type 2 diabetes)⁵⁰. HGF was also reported as causally linked with BMI⁵¹. Furthermore, a list of 18 proteins found to be independently associated with cardiovascular death included HGF and SPON1¹³. Each protein may therefore have an independent function related to disease, but the biosignature protein combination may be indicative of a specific disease activity.

To understand if the biosignature is relevant to patient outcomes, we explored its presence in studies where the biosignature proteins have been measured and where outcome data are available. In a study of patients that were hospitalised due to COVID-19-related symptoms, 11 of the 384 patients exhibited the biosignature at day 0, upon admission to hospital, and had higher death rates compared to patients without the biosignature. A similar observation was made in a smaller cohort of patients with ESKD and COVID-19 (subcohort A), where distinctly higher mortality rates occurred in those with the biosignature, compared with patients without the biosignature. In a second small ESKD COVID-19 cohort (subcohort B) those with the biosignature had severe or moderate disease and survived. Patients in subcohort B were admitted to hospital at later timepoints when their disease had progressed much further than in subcohort A, and had serum analysed rather than plasma, which may account for differences observed between the two subcohorts. Furthermore, the ESKD cohorts had higher median levels for the biosignature proteins, particularly HGF. If the biosignature is further validated in other relevant cohorts, it may be appropriate to apply different absolute protein thresholds for identifying individuals with biosignature depending on the underlying disease. These studies suggest that patients exhibiting the biosignature have poor outcomes and may be more vulnerable when exposed to specific triggers, including viral infection.

The discovery of the systemic biosignature in our initial studies was unexpected, and was uncovered while comparing individual patient coronary and systemic protein levels. Few other studies have compared coronary artery and systemic blood. Chandran et al²⁶ used an aspiration catheter to collect coronary blood samples from patients with ST-segment elevation myocardial infarction (STEMI), and separated thrombus particulate matter from blood using filtration. In their study, the aim was to identify inflammatory proteins in coronary and systemic blood samples that linked with plaque erosion or rupture. Although coronary and systemic samples were not compared directly, it was interesting to observe that HGF mRNA expression levels were identified as significantly associating with ruptured plaques (compared with eroded plaques). However, this observation was not observed in systemic samples, possibly indicating that coronary sampling has advantages over systemic sampling in detecting coronary disease-related factors. Additionally, coronary catheter-sampled blood had higher levels of CXCL9 in patients that went on to have major adverse cardiac events (MACE), compared with absence of MACE in those with lower levels of CXCL9⁵².

The raised levels of these proteins may be temporal in nature, either due to local changes causing release of proteins from the endothelial glycocalyx, damaged cells or activated neutrophils or mast cells, or an event such as inflammation causes their increased de novo synthesis. Activated mast cells and neutrophils have roles in development of atherosclerosis and if the biosignature reflects active atherosclerotic inflammation and repair, could this blood vessel activity explain the increased vulnerability to severe illness when exposed to pathogens such as SARS-Cov-2? In support of this notion, mast cell activity was linked with disease progression in COVID-19 patients³⁵. Of particular relevance, mast cells contain heparin⁵³ and mast cell activation leading to the release of heparin could induce increased blood levels of the biosignature proteins via release from the endothelial glycocalyx. Increased mast cell activity, as measured by serum tryptase levels, was reported as an indicator of poor CVD outcomes and interestingly, tryptase levels did not correlate with CRP levels in this study, suggesting CRP is not a surrogate marker for mast cell activity⁵⁴. In the present study, individuals with the biosignature in the cardiovascular cohorts had low-mid CRP levels (<5 mg/L), indicating a low to moderate mortality risk⁵⁵. In the COVID-19 cohorts, patients had a broad range of CRP values (from 0.6 to 360 mg/L). In each cohort investigated, the biosignature protein levels did not correlate with CRP levels, except in the ESKD cohorts where CRP correlated positively and significantly with HGF³⁰. However, in ESKD subcohort A, patients with the biosignature had higher CRP levels compared with the remaining cohort. Our study raises the possibility that knowledge of CRP levels combined with measurements of biosignature protein levels could provide individuals with a broader picture of cardiovascular inflammation and better risk prediction.

The biosignature may have gone undetected in previous studies for various reasons including that it appears to be expressed in a minority of individuals. Another possibility is that a few patients with very high levels for certain proteins that cannot be otherwise explained may be excluded from analysis as outliers.

Although outcome data is not currently available for CVD cohort studies, a clear and significant difference was observed in mortality of patients with the biosignature in the respiratory disease/COVID-19 cohort. The finding of increased mortality in a subcohort of COVID-19 positive ESKD patients with the biosignature provided validation for the association of the biosignature with poor outcomes. The strengths of the present study are that we identified a biosignature in a small CVD cohort and verified these findings in a larger independent CVD cohort and in cohorts with more complicated disease profiles.

In conclusion, a systemic biosignature was detected in patients with known or suspected cardiovascular disease and a mid-level of calcification, indicative of the presence of atherosclerosis and a moderate risk of future cardiovascular events. What the biosignature might indicate is yet to be determined in future studies with sufficient power to test if protein levels can be linked with cardiovascular outcomes. From analysis of data from patients in COVID-19 cohorts it appears to be predictive of worse outcomes, possibly due to pre-existing CVD. The biosignature has the potential to be developed as a simple, non-invasive systemic blood test indicative of active vascular damage/repair that may be related to mast cell activation, neutrophil activity or endogenous heparin release. As such, we can speculate its use to indicate active disease in asymptomatic individuals, altering prediction of adverse outcomes for cardiovascular disease patients or with pathogen infections (e.g., SARS-Cov-2). Although several biomolecules have been identified that link with cardiovascular risk, very few have been adopted for routine clinical use.

### Example 2—Identification of the Biosignature in the IMPROVE Cohort

### Background

The IMPROVE cohort has 3711 participants with at least 3 established CVD risk factors (men, post-menopausal women, dyslipidaemia, hypertension, diabetes, smoking and family history of CVD) but with no overt cardiovascular disease at the time of recruitment^{66,67}. After blood sampling and carotid measurements were taken at baseline, 3372 participants were included in the analysis and followed up after 3 years. 192 cardiovascular events were recorded at 3-year follow-up, defined as myocardial infarction, ischemic stroke, peripheral artery disease or revascularization procedures. Proteomic analysis of plasma was performed using the CVD1 panel at Olink (Sweden), which contains 3 of the proteins in the biosignature: HGF, PAPPA and SPON-1.

### Aim

The inventors wished to establish if any of the participants in the IMPROVE cohort had the biosignature, and if so, did these participants differ from the main cohort in terms of carotid disease parameters or cardiovascular outcomes.

### Results of Analysis of the Biosignature in IMPROVE

All statistical analysis was performed by Dr. Bruna Gigante using STATA v14 and summarized in the Table below. The biosignature was defined as ≥90^{th} centile levels for HGF, PAPPA and SPON-1, which identified 41 participants—a small minority (1.2%) of the whole cohort and comprised of 28 men and 13 women. Using linear logistic regression analysis to compare the carotid-intima media thickness (c-IMT) of the biosignature group with the remaining cohort (<90th centile) revealed a borderline statistically significant increase in c-IMTₘₐₓ in the biosignature group (P=0.073). The biosignature proteins displayed a positive and significant correlation with each other (Pearson coefficient pairwise correlations: R≥0.34, P<0.0001). No correlation was observed between biosignature proteins and CRP levels. However, the biosignature group had higher CRP levels (median 3.1 mg/L, range 2.2-4.4 mg/L) compared with the remaining cohort (median 1.8 mg/L, range 0.76-3.54 mg/L).

8 cardiovascular events occurred in the group with the biosignature, i.e., 8/41, approximately 20%. Using a Cox regression model, participants with the biosignature had a significantly increased risk of future cardiovascular events with an HR of 3.08 and 95% CI (1.51-6.29), p=0.002.

### Conclusions

41 individuals with the biosignature were detected in the IMPROVE cohort. These participants had a higher maximum c-IMT level compared with participants without the biosignature, suggesting the biosignature group of individuals may be at higher risk of cardiovascular events, but this observation was borderline significant.

The biosignature group had a statistically significant increase in the risk of cardiovascular events, suggesting that the biosignature indicates poorer outcomes. However, the large confidence interval needs to be noted.

Although only 3 of the biosignature proteins were measured in IMPROVE, this was sufficient to identify participants with the biosignature pattern. Analysis in the IMPROVE cohort supports findings made in CS1, PACIFIC, respiratory disease/COVID-19 cohort and a CKD/COVID-19 cohort—that individuals with the biosignature are:
a minority of individuals with suspected or known CVD
male or female, with some cohorts suggesting more common in males
at increased risk of poor outcomes

### REFERENCES

1. Nowbar et al., Circ Cardiovasc Qual Outcomes 12, e005375, doi:10.1161/CIRCOUTCOMES.118.005375 (2019).
2. Baigent et al., Lancet 366, 1267-1278, doi:10.1016/S0140-6736(05)67394-1 (2005).
3. Yao et al., Nat Commun 9, 3268, doi:10.1038/s41467-018-05512-x (2018).
4. Klarin et al., Nat Genet 50, 1514-1523, doi:10.1038/s41588-018-0222-9 (2018).
5. Kraus et al., PLOS Genet 11, e1005553, doi:10.1371/journal.pgen.1005553 (2015).
6. Libby & Ebert, Circulation 138:666-668, doi:10.1161/CIRCULATIONAHA.118.034392 (2018).
7. Tazaki et al. Int Heart J 57:18-24, doi:10.1536/ihj.15-248 (2016).
8. Chai et al., J Geriatr Cardiol 14:285-291 doi:10.11909/j.issn.1671-5411.2017.05.013 (2017).
9. Bom et al. EBioMedicine 39, 109-117, doi:10.1016/j.ebiom.2018.12.033 (2019).
10. Havulinna et al., Arterioscler Thromb Vasc Biol 36, 2424-2430, doi:10.1161/ATVBAHA.116.307497 (2016).
11. Poss et al., J Clin Invest 130, 1363-1376, doi:10.1172/JCI131838 (2020).
12. Lepojarvi et al., Am J Cardiol 117:515-521, doi:10.1016/j.amjcard.2015.11.038 (2016).
13. Wallentin et al., PLOS Med 18:e1003513, doi:10.1371/journal.pmed.1003513 (2021).
14. Rossini et al., Catheter Cardiovasc Interv 85: E129-139, doi:10.1002/ccd.25724 (2015).
15. Greenland, J Am Coll Cardiol 72:434-447, doi:10.1016/j.jacc.2018.05.027 (2018).
16. Budoff et al., J Am Coll Cardiol 49:1860-1870, doi:10.1016/j.jacc.2006.10.079 (2007).
17. Antoniades et al., Eur Heart J 41:748-758, doi:10.1093/eurheartj/ehz474 (2020).
18. Ridker et al., N Engl J Med 377: 1119-1131, doi:10.1056/NEJMoa1707914 (2017).
19. Ridker et al., Eur Heart J, doi:10.1093/eurheartj/ehaa160 (2020).
20. Aday & Ridker, Front Cardiovasc Med 6:16, doi:10.3389/fcvm.2019.00016 (2019).
21. Ridker et al., Lancet 391:319-328, doi:10.1016/S0140-6736(17)32814-3 (2018).
22. de Kleijn et al., Arterioscler Thromb Vasc Biol 30:612-619, doi:10.1161/ATVBAHA.109.194944 (2010).
23. Lee et al., Clin Proteomics 14:22, doi:10.1186/s12014-017-9157-x (2017).
24. Alonso-Orgaz et al., J Proteom 109, 368-381, doi:10.1016/j.jprot.2014.07.016 (2014).
25. Yong et al., Blood 117:11-20, doi:10.1182/blood-2010-04-278812 (2011).
26. Chandran et al., J Am Heart Assoc 6, doi:10.1161/JAHA.117.005868 (2017).
27. Truong et al., Heart Rhythm 11:2167-2175, doi:10.1016/j.hrthm.2014.07.007 (2014).
28. West et al., JACC: Basic to Translational Science 2:646-654, doi:10.1016/j.jacbts.2017.07.007 (2017).
29. Filbin et al., Cell Rep Med 2:100287, doi:10.1016/j.xcrm.2021.100287 (2021).
30. Gisby et al., Elife 10, doi:10.7554/eLife.64827 (2021).
31. Uhlen et al., Sci Signal 12, doi:10.1126/scisignal.aaz0274 (2019).
32. Ngo et al., Circulation 134:270-285, doi:10.1161/CIRCULATIONAHA.116.021803 (2016).
33. Beck et al., Clin Chem 64:1474-1484, doi:10.1373/clinchem.2017.282665 (2018).
34. Abildgaard, Haemostasis 23 Suppl 1:103-106, doi:10.1159/000216918 (1993).
35. Salbach et al., Br J Clin Pharmacol 50:221-226, doi:10.1111/j.1365-2125.2000.00246.x (2000).
36. Terkelsen et al., Am J Cardiol 103:29-35, doi:10.1016/j.amjcard.2008.08.027 (2009).
37. Ranjbaran et al., Circulation 114:1293-1300, doi:10.1161/CIRCULATIONAHA.106.631457 (2006).
38. Bell et al. Atherosclerosis 272:162-167, doi:10.1016/j.atherosclerosis.2018.03.040 (2018).
39. Li et al., Arch Med Sci 9:389-397, doi:10.5114/aoms.2013.35421 (2013).
40. Palmer et al., PloS one 9:e98388, doi:10.1371/journal.pone.0098388 (2014).
41. Brunner et al., Sci Rep 7:8707, doi:10.1038/s41598-017-09207-z (2017).
42. Diaz-Lopez et al., Cardiovasc Diabetol 13:51, doi:10.1186/1475-2840-13-51 (2014).
43. Winckers et al. J Thromb Haemost 9:2243-2250, doi:10.1111/j. 1538-7836.2011.04497.x (2011).
44. Morange et al., J Thromb Haemost 5:475-482, doi:10.1111/j. 1538-7836.2007.02372.x (2007).
45. Klimczak-Tomaniak et al., ESC Heart Failure 7:1190-1200, doi:https://doi.org/10.1002/ehf2.12678 (2020).
46. Malatino et al., Am J Kidney Dis 36:945-952, doi:10.1053/ajkd.2000.19087 (2000).
47. Liu et al., Nal Sci Rev 7:1003-1011, doi:10.1093/nsr/nwaa037 (2020).
48. Deng et al., medRxiv, 2020.2005.2031.20118315, doi:10.1101/2020.05.31.20118315 (2020).
49. Meizlish et al., Blood Adv 5:1164-1177, doi:10.1182/bloodadvances.2020003568 (2021).
50. Folkersen et al., Nat Metab 2:1135-1148, doi:10.1038/s42255-020-00287-2 (2020).
51. Karhunen et al., medRxiv, 2020.2010.2026.20219477, doi:10.1101/2020.10.26.20219477 (2020).
52. Mccarthy et al., Am J Cardiol 120:25-32, doi:10.1016/j.amjcard.2017.03.265 (2017).
53. da Silva et al., J Histochem Cytochem 62:698-738, doi:10.1369/0022155414545334 (2014).
54. Morici et al., Open Heart 3:e000472, doi:10.1136/openhrt-2016-000472 (2016).
55. Kaura et al., PLOS Med 19:e1003911, doi:10.1371/journal.pmed.1003911 (2022).
56. Decker et al., Am Heart J 213:30-34, doi:10.1016/j.ahj.2019.03.018 (2019).
57. Santalahti et al., J Intern Med 282:340-352, doi:10.1111/joim.12648 (2017).
58. Haljasmägi et al., Sci Rep 10:20533, doi:10.1038/s41598-020-77525-w (2020).
59. Bayes-Genis et al., N Engl J Med 345: 1022-1029, doi:10.1056/NEJMoa003147 (2001).
60. Nilsson et al., J Clin Med 9, doi:10.3390/jcm9010265 (2020).
61. Bernardi et al., Clin Sci (Lond) 133:1145-1166, doi:10.1042/CS20181116 (2019).
62. McMahon et al., Arthritis Rheumatol 66:130-139, doi:10.1002/art.38204 (2014).
63. Turkmen et al., BMC Nephrol 14:144-144, doi:10.1186/1471-2369-14-144 (2013).
64. Morange et al., J Thromb Haemost 5:475-482, doi:10.1111/j.1538-7836.2007.02372.x (2007).
65. Barska et al., PloS One 12:e0181533-e0181533, doi:10.1371/journal.pone.0181533 (2017).
66. Baldassarre et al., Eur Heart J 31:614-622, doi:10.1093/eurheartj/ehp496 (2010).
67. Baldassarre et al., J Am Coll Cardiol 60:1489-1499, doi:10.1016/j.jacc.2012.06.034 (2012).
68. Kubo et al., J Dermatol Sci 93:33-40, doi:10.1016/j.jdermsci.2018.10.001 (2019).
69. Rehman et al., J Am Coll Cardiol 41:1408-1413, doi:https://doi.org/10.1016/S0735-1097(03)00231-6 (2003).
70. Peña-Silva et al., Hypertension 65:587-593, doi:10.1161/HYPERTENSIONAHA.114.04681 (2015).
71. Swiger et al., Amyloid 23:242-248, doi:10.1080/13506129.2016.1242480 (2016).
72. Santalahti et al., J Intern Med 282:340-352, doi:https://doi.org/10.1111/joim. 12648 (2017).
73. Surbone et al., BMC Research Notes 5:517, doi:10.1186/1756-0500-5-517 (2012).
74. Song et al., Clin Proteom 16:10-10, doi:10.1186/s12014-019-9230-8 (2019).
75. Coskun et al., J Cardiol 61:128-131, doi:https://doi.org/10.1016/j.jjcc.2012.09.007 (2013).
76. Kløverpris et al., Mol Hum Reprod 19:756-763, doi:10.1093/molehr/gat047 (2013).
77. Maarouf et al., J Trans Med 17:51, doi:10.1186/s12967-019-1789-3 (2019).
78. Martin-Ventura et al., Atherosclerosis 214:486-489, doi:10.1016/j.atherosclerosis.2010.11.009 (2011).
79. Guo et al., Emerg Med Int 2019, 7238705, doi:10.1155/2019/7238705 (2019).
80. Carrero et al., Clin J Am Soc Nephrol 4, 110-118, doi:10.2215/CJN.02790608 (2009).
81. Yamamuro et al., Blood Coagul & Fibrinolysis 9:491-497, doi:10.1097/00001721-199809000-00006 (1998).
82. Gu et al., Blood 126:4672-4672, doi:10.1182/blood.V126.23.4672.4672 (2015).
83. Dahm et al., Blood 101:4387-4392, doi:10.1182/blood-2002-10-3188 (2003).
84. Ezzat et al., Int J Dermatol 48:822-829, doi:https://doi.org/10.1111/j.1365-4632.2009.04069.x (2009).
85. Kagami et al., J Invest Dermatol 124: 1088-1090, doi:10.1111/j.0022-202X.2005.23700.x (2005).

The foregoing embodiments, instances, and examples are applicable to any of the aspects of the present disclosure and should be construed as such.

The listing or discussion of an apparently prior-published document in this specification should not necessarily be taken as an acknowledgement that the document is part of the state of the art or is common general knowledge.

While the present disclosure has been described in terms of various aspects, embodiments, and examples, it is understood that variations, improvements, and equivalents will occur to the person skilled in the art. Such variations, improvements, and equivalents are contemplated by the present disclosure and fall within the scope of the matter disclosed and claimed herein.

1. A method for identifying if a patient is predisposed to experiencing a severe episode of a disease and/or at risk of experiencing a severe episode of a disease, comprising the steps of:
i) detecting the presence of a biosignature comprising the biomarkers hepatocyte growth factor (HGF) and Spondin-1 (SPON-1) in the patient's blood, and
ii) identifying that the patient is predisposed to experiencing a severe episode of a disease by the presence of the biosignature and/or identifying that the patient is at risk of experiencing a severe episode of a disease by the presence of the biosignature.

2. (canceled)

3. A method of evaluating the effectiveness of a medical intervention in a patient, comprising the steps of:
a) detecting the presence of a biosignature comprising the biomarkers hepatocyte growth factor (HGF) and Spondin-1 in the patient's blood at a first time,
b) applying the medical intervention to the patient,
c) detecting the presence of said biosignature comprising the biomarkers hepatocyte growth factor (HGF) and Spondin-1 (SPON-1) in the patient's blood at a second time, and
d) evaluating the effectiveness of the medical intervention by comparing the presence of the biosignature comprising the biomarkers in step a) with the presence of said biosignature comprising the biomarkers in step c).

4. A method of selecting a medical intervention for a patient, comprising the steps of:
i) detecting the presence of a biosignature comprising the biomarkers hepatocyte growth factor (HGF) and Spondin-1 in the patient's blood,
ii) identifying that the patient is predisposed to experiencing a severe episode of a disease by the presence of the biosignature and/or identifying that the patient is at risk of experiencing a severe episode of a disease by the presence of the biosignature, and
iii) selecting a medical intervention for the patient.

5. A method according to claim 4, additionally comprising:
iv) administering the medical intervention to the patient,
such that a severe episode of a disease in a patient is treated and/or prevented.

6. The method of claim 1, wherein the method or use further comprises before step i) the step of:
i(i) providing a blood sample from the patient; and
wherein the detecting the presence of the biosignature in the patient's blood in step i) comprises detecting the presence of the biosignature in the sample of step i(i).

7. The method according to claim 3, wherein the method further comprises before step (a) the step of:
a(i) providing a first blood sample from the patient; and
wherein the detecting the presence of the biosignature in the patient's blood at a first time in step (a) comprises detecting the presence of the biosignature in the sample of step a(i); and wherein the method further comprises before step (c) the step of:
c(i) providing a second blood sample from the patient; and
wherein the detecting the presence of the biosignature in the patient's blood at a second time in step (c) comprises detecting the presence of the biosignature in the sample of step c(i).

8. The method of claim 1, wherein the biosignature further comprises one or more biomarkers selected from the group consisting of: TNF-related weak inducer of apoptosis (TWEAK); tissue factor pathway inhibitor (TFPI); pappalysin-1 (PAPPA); CCL28; CXCL9; and Azu-1; or any combination thereof.

9. The method of claim 1, wherein the biosignature comprises all of: hepatocyte growth factor (HGF); TNF-related weak inducer of apoptosis (TWEAK); Spondin-1 (SPON-1); tissue factor pathway inhibitor (TFPI); pappalysin-1 (PAPPA); CCL28; and CXCL9.

10. The method of claim 6, wherein the blood sample is a systemic blood sample, such as a peripheral blood sample.

11. (canceled)

12. The method or use of claim 1, wherein the disease is a disease associated with inflammation.

13. (canceled)

14. The method of claim 12, wherein the inflammation is vascular inflammation or is associated with endothelial damage.

15. (canceled)

16. (canceled)

17. The method of claim 1, wherein the disease is a cardiovascular disease or an infectious disease.

18. The method of claim 1, wherein the disease is not associated with general systemic inflammation.

19. The method of claim 18, wherein the general systemic inflammation is characterised by the presence of the biomarker C-reactive protein (CRP).

20. The method of claim 19, wherein step i) further comprises detecting the presence of the biomarker C-reactive protein (CRP) in the patient's blood, and step ii) further comprises identifying that the disease is not associated with general systemic inflammation by the presence of a low level of CRP.

21. The method of claim 1, wherein the severe episode of the disease is characterised by the patient needing a medical intervention to recover from the episode of the disease and/or the patient being at risk of death due to the episode of the disease.

22. (canceled)

23. The method of claim 21, wherein the severe episode is a major adverse coronary event (MACE).

24. (canceled)

25. (canceled)

26. (canceled)

27. The method of claim 1, wherein detecting the presence of the biosignature comprises detecting an elevated level of the biomarkers comprised by the biosignature.

28. The method of claim 27, wherein the elevated level of the biomarkers is elevated when compared to the level of the biomarkers in blood from an individual that is not predisposed to experiencing a severe episode of a disease and/or at risk of experiencing a severe episode of a disease.

29. The method of claim 28, wherein the elevated level of each biomarker:
a) is a two-fold or more increase; and/or
b) is in the 90^{th} percentile of the level of the biomarker in a reference population and >1.5 times the median level of the biomarker in a reference population.

30. (canceled)

31. (canceled)

32. (canceled)
